(19) United States
(12) Patent Application Publication
(10) Pub. No.: US 2024/0420827 A1
(43) Pub. Date: Dec. 19, 2024

(54) IMAGE PROCESSING APPARATUS, ENDOSCOPE APPARATUS, IMAGE PROCESSING METHOD, AND PROGRAM

(71) Applicant: FUJIFILM Corporation, Tokyo (JP)

(72) Inventor: Yoshinori ITAI, Kanagawa (JP)

(73) Assignee: FUJIFILM Corporation, Tokyo (JP)

(21) Appl. No.: 18/819,999

(22) Filed: Aug. 29, 2024

Related U.S. Application Data
(63) Continuation of application No. PCT/JP2023/004986, filed on Feb. 14, 2023.

(30) Foreign Application Priority Data
Mar. 2, 2022 (JP) 2022-032001

(51) Int. Cl. G16H 30/20; G06F 3/14; G06T 5/30; G16H 30/40

(52) U.S. Cl. CPC G16H 30/20; G06F 3/14; G06T 5/30; G16H 30/40

(57) ABSTRACT

An image processing apparatus includes a processor. The processor displays, on a display device, a moving image that is generated based on volume data including a bronchial image showing a bronchus and that shows an aspect in which an inside of the bronchus is observed, receives an instruction performed by a voice, acquires a positional relationship between a plurality of positions from an upstream side to a downstream side in the bronchus according to the instruction, and displays the moving image in a display aspect corresponding to the positional relationship.

### CROSS-REFERENCE TO RELATED APPLICATIONS

This application is a continuation application of International Application No. PCT/JP2023/004986, filed Feb. 14, 2023, the disclosure of which is incorporated herein by reference in its entirety. Further, this application claims priority under 35 USC 119 from Japanese Patent Application No. 2022-032001 filed Mar. 2, 2022, the disclosure of which is incorporated by reference herein.

### BACKGROUND

### 1. Technical Field

The technology of the present disclosure relates to an image processing apparatus, an endoscope apparatus, an image processing method, and a program.

### 2. Related Art

JP2006-223374A discloses a surgical support apparatus including image processing means for processing a virtual image of a surgical part to create a processed image, and processed image recording means for recording the processed image created by the image processing means according to a progress process of an endoscopic surgery on the surgical part.

In addition, the surgical support apparatus disclosed in JP2006-223374A further includes display control means. In the surgical support apparatus disclosed in JP2006-223374A, the virtual image recording means records a non-processed virtual image of the surgical part along with the processed image, and the display control means reads out the processed image or the virtual image and displays the read out processed image or virtual image on display means according to the progress process of the endoscopic surgery.

### SUMMARY

One embodiment according to the technology of the present disclosure provides an image processing apparatus, an endoscope apparatus, an image processing method, and a program which can display a moving image in a display aspect convenient for an observer.

A first aspect according to the technology of the present disclosure relates to an image processing apparatus comprising: a processor, in which the processor displays, on a display device, a moving image that is generated based on volume data including a bronchial image showing a bronchus and that shows an aspect in which an inside of the bronchus is observed, receives an instruction performed by a voice, acquires a positional relationship between a plurality of positions from an upstream side to a downstream side in the bronchus according to the instruction, and displays the moving image in a display aspect corresponding to the positional relationship.

A second aspect according to the technology of the present disclosure relates to the image processing apparatus according to the first aspect, in which the plurality of positions include a position corresponding to an upstream bifurcation in the bronchus and a position corresponding to a downstream bifurcation in the bronchus.

A third aspect according to the technology of the present disclosure relates to the image processing apparatus according to the second aspect, in which the position corresponding to the upstream bifurcation is a position upstream of the upstream bifurcation in the bronchus, and the position corresponding to the downstream bifurcation is a position upstream of the downstream bifurcation in the bronchus.

A fourth aspect according to the technology of the present disclosure relates to the image processing apparatus according to the third aspect, in which the display aspect includes an aspect in which the display of the moving image is stopped at the position corresponding to the upstream bifurcation and at the position corresponding to the downstream bifurcation, an aspect in which a speed at which the display of the moving image is advanced is decreased, or an aspect in which the display of the moving image is stopped after the speed at which the display of the moving image is advanced is decreased.

A fifth aspect according to the technology of the present disclosure relates to the image processing apparatus according to any one of the first to fourth aspects, in which the positional relationship is defined by a first distance that is a distance between the plurality of positions.

A sixth aspect according to the technology of the present disclosure relates to the image processing apparatus according to the fifth aspect, in which the display aspect includes an aspect in which the display of the moving image is advanced at a speed determined according to a time required for movement between the plurality of positions and the first distance.

A seventh aspect according to the technology of the present disclosure relates to the image processing apparatus according to any one of the first to sixth aspects, in which the display aspect includes a rotational display aspect in which the moving image is rotationally displayed around a rotation axis determined for the moving image according to the positional relationship.

An eighth aspect according to the technology of the present disclosure relates to the image processing apparatus according to the seventh aspect, in which the rotation axis is obtained by thinning the bronchial image.

A ninth aspect according to the technology of the present disclosure relates to the image processing apparatus according to the seventh or eighth aspect, in which a speed at which the moving image is rotationally displayed is determined according to the positional relationship.

A tenth aspect according to the technology of the present disclosure relates to the image processing apparatus according to the ninth aspect, in which the positional relationship is defined by a second distance that is a distance between the plurality of positions, and the speed at which the moving image is rotationally displayed is determined according to the second distance and a time required for movement between the plurality of positions.

An eleventh aspect according to the technology of the present disclosure relates to the image processing apparatus according to any one of the seventh to tenth aspects, in which the moving image shows an aspect in which the inside of the bronchus is observed from a viewpoint in the bronchus, and the rotational display aspect is an aspect in which the moving image is rotationally displayed around the rotation axis in response to movement of the viewpoint between the plurality of positions, to rotate an opening portion image region showing an opening portion of a bifurcation included in the bronchus toward an upper side in a front view of the display region in which the moving image is displayed.

A twelfth aspect according to the technology of the present disclosure relates to the image processing apparatus according to the eleventh aspect, in which the rotational display aspect is an aspect in which the opening portion image region is rotated by a rotation amount corresponding to an angle about the rotation axis between a position of the opening portion image region and a target position on the upper side in the front view.

A thirteenth aspect according to the technology of the present disclosure relates to the image processing apparatus according to the eleventh or twelfth aspect, in which the rotational display aspect is an aspect in which the moving image is rotationally displayed around the rotation axis, to locate the opening portion image region on the upper side in the front view at a timing at which the viewpoint reaches a termination position among the plurality of positions.

A fourteenth aspect according to the technology of the present disclosure relates to the image processing apparatus according to any one of the first to thirteenth aspects, in which the processor does not receive the instruction or ignores the received instruction during a period in which the moving image is displayed in the display aspect.

A fifteenth aspect according to the technology of the present disclosure relates to the image processing apparatus according to any one of the first to fourteenth aspects, in which the moving image shows an aspect in which the inside of the bronchus is observed along a pathway obtained by thinning the bronchial image.

A sixteenth aspect according to the technology of the present disclosure relates to the image processing apparatus according to the fifteenth aspect, in which the display aspect includes an aspect in which the display of the moving image is advanced at a constant speed along the pathway between the plurality of positions.

A seventeenth aspect according to the technology of the present disclosure relates to an endoscope apparatus comprising: the image processing apparatus according to any one of the first to sixteenth aspects; and an endoscope that acquires an image showing an aspect of the inside of the bronchus by imaging the inside of the bronchus and that outputs the acquired image.

An eighteenth aspect according to the technology of the present disclosure relates to an image processing method comprising: displaying, on a display device, a moving image that is generated based on volume data including a bronchial image showing a bronchus and that shows an aspect in which an inside of the bronchus is observed; receiving an instruction performed by a voice; acquiring a positional relationship between a plurality of positions from an upstream side to a downstream side in the bronchus according to the instruction; and displaying the moving image in a display aspect corresponding to the positional relationship.

A nineteenth aspect according to the technology of the present disclosure relates to a program causing a computer to execute a process comprising: displaying, on a display device, a moving image that is generated based on volume data including a bronchial image showing a bronchus and that shows an aspect in which an inside of the bronchus is observed; receiving an instruction performed by a voice; acquiring a positional relationship between a plurality of positions from an upstream side to a downstream side in the bronchus according to the instruction; and displaying the moving image in a display aspect corresponding to the positional relationship.

### BRIEF DESCRIPTION OF THE DRAWINGS

Exemplary embodiments of the technology of the disclosure will be described in detail based on the following figures, wherein:

FIG. 1 is a conceptual diagram illustrating an example of an aspect in which an endoscope system is used;

FIG. 2 is a conceptual diagram illustrating an example of an overall configuration of the endoscope system;

FIG. 3 is a conceptual diagram illustrating an example of an aspect in which an insertion part of a bronchoscope is inserted into a body of a subject;

FIG. 4 is a block diagram illustrating an example of a hardware configuration of an endoscope apparatus;

FIG. 5 is a block diagram illustrating an example of main functions of a processor;

FIG. 6 is a conceptual diagram illustrating an example of a configuration of a virtual image generation device;

FIG. 7 is a conceptual diagram illustrating contents of a virtual image generation process performed by the virtual image generation device and an example of contents of a process of a first display control unit;

FIG. 8 is a conceptual diagram illustrating contents of the virtual image generation process performed by the virtual image generation device and an example of contents of a process of a virtual bronchial moving image acquisition unit;

FIG. 9 is a conceptual diagram illustrating an example of contents of processes of a second display control unit and a third display control unit;

FIG. 10 is a conceptual diagram illustrating an example of contents of processes of the third display control unit, a voice recognition unit, and a positional relationship acquisition unit.

FIG. 11 is a conceptual diagram illustrating an example of contents of processes of a display aspect determination unit and the third display control unit;

FIG. 12 is a flowchart illustrating an example of a flow of a virtual bronchial moving image acquisition process;

FIG. 13 is a flowchart illustrating an example of a flow of an endoscopic image display process;

FIG. 14 is a flowchart illustrating an example of a flow of a virtual bronchial moving image display process;

FIG. 15 is a conceptual diagram illustrating a modification example of an aspect in which display of a virtual bronchial moving image is advanced;

FIG. 16 is a conceptual diagram illustrating an example of a rotational display aspect;

FIG. 17 is a conceptual diagram illustrating a modification example of the rotational display aspect;

FIG. 18 is a conceptual diagram illustrating a form example in which a voice instruction is ignored; and

FIG. 19 is a conceptual diagram illustrating an example of an aspect in which a current position of a viewpoint is displayed by a seek bar.

### DETAILED DESCRIPTION

In the following, an example of embodiments of an image processing apparatus, an endoscope apparatus, an image processing method, and a program according to the technology of the present disclosure will be described with reference to accompanying drawings. The terms used in the following description will be described first.

CPU is an abbreviation for “central processing unit”. GPU is an abbreviation for “graphics processing unit”. RAM is an abbreviation for “random access memory”. NVM is an abbreviation for “non-volatile memory”. EEPROM is an abbreviation for “electrically erasable programmable read-only memory”. ASIC is an abbreviation for “application specific integrated circuit”. PLD is an abbreviation for “programmable logic device”. FPGA is an abbreviation for “field-programmable gate array”. SoC is an abbreviation for “system-on-a-chip”. SSD is an abbreviation for “solid state drive”. USB is an abbreviation for “universal serial bus”. HDD is an abbreviation for “hard disk drive”. EL is an abbreviation for “electro-luminescence”. I/F is an abbreviation for “interface”. CMOS is an abbreviation for “complementary metal oxide semiconductor”. CCD is an abbreviation for “charge coupled device”. CT is an abbreviation for “computed tomography”. MRI is an abbreviation for “magnetic resonance imaging”.

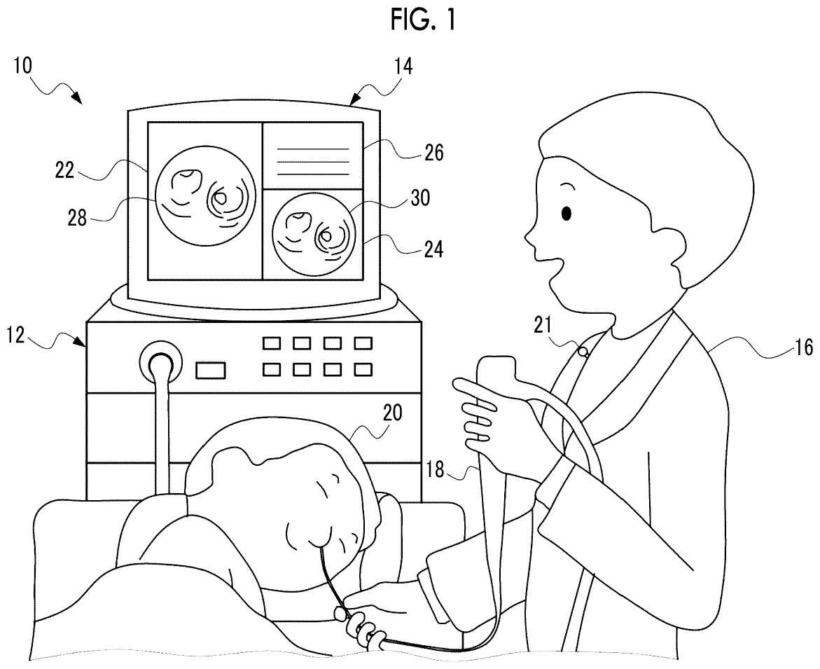

As an example, as illustrated in FIG. 1, an endoscope system 10 comprises an endoscope apparatus 12 and a display device 14. The endoscope apparatus 12 is used by a medical practitioner (hereinafter, referred to as a “user”) such as a doctor 16, a nurse, and/or a technician. The endoscope apparatus 12 is an apparatus that comprises a bronchoscope 18 and performs a medical treatment on a bronchus of a subject 20 (for example, a patient) via the bronchoscope 18. The endoscope apparatus 12 is an example of an “endoscope apparatus” according to the technology of the present disclosure, and the bronchoscope 18 is an example of an “endoscope” according to the technology of the present disclosure.

The bronchoscope 18 is inserted into the bronchus of the subject 20 by the doctor 16, images an inside of the bronchus, acquires an image showing an aspect of the inside of the bronchus, and outputs the acquired image. In the example illustrated in FIG. 1, an aspect in which the bronchoscope 18 is inserted into a body through a nostril of the subject 20 is illustrated. It should be noted that, in the example illustrated in FIG. 1, although the bronchoscope 18 is inserted into the body through the nostril of the subject 20, this is merely an example, and the bronchoscope 18 may be inserted into the body through a mouth of the subject 20.

The endoscope apparatus 12 comprises a microphone 21. The microphone 21 acquires a voice uttered by the doctor 16, and outputs a voice signal indicating the acquired voice to a predetermined output destination. Examples of the microphone 21 include a pin microphone. In the example illustrated in FIG. 1, the microphone 21 is attached to a collar of the doctor 16. It should be noted that the microphone 21 need only be installed at a position at which the voice of the doctor 16 can be acquired, and a microphone having directivity toward the mouth of the doctor 16 is preferable. In the example illustrated in FIG. 1, the pin microphone is given as an example of the microphone 21. However, this is merely an example, and the microphone 21 may be other types of microphones such as a stand microphone and a bone conduction microphone.

The display device 14 displays various types of information including an image. Examples of the display device 14 include a liquid crystal display and an EL display. A plurality of screens are displayed side by side on the display device 14. In the example illustrated in FIG. 1, a first screen 22, a second screen 24, and a third screen 26 are illustrated as examples of the plurality of screens.

An endoscopic image 28 obtained by imaging the bronchus of the subject 20 via the bronchoscope 18 is displayed on the first screen 22. Examples of the endoscopic image 28 include a moving image (for example, a live view image). A virtual image 30 is displayed on the second screen 24. Examples of the virtual image 30 include a moving image. The virtual image 30 is a virtual image showing an aspect in which an inside of a virtual bronchus simulating the bronchus observed by the doctor 16 through the endoscopic image 28 is observed from a virtually set viewpoint. For example, information on the subject 20 and/or information on an operation of the endoscope apparatus 12 is displayed on the third screen 26.

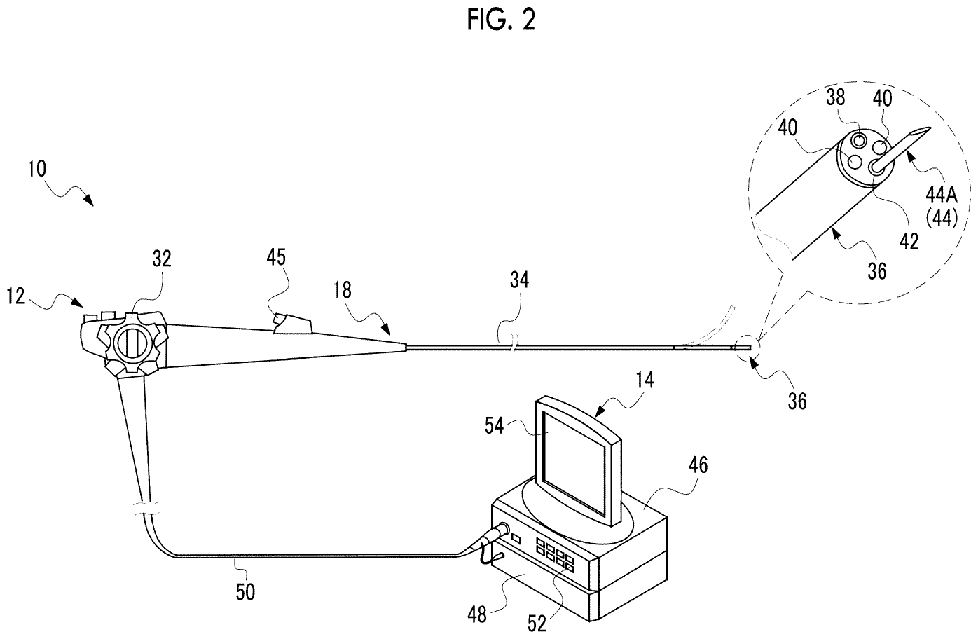

As an example, as illustrated in FIG. 2, the bronchoscope 18 comprises an operating part 32 and an insertion part 34. The insertion part 34 is formed in a tubular shape. An outer contour of the insertion part 34 in a cross-sectional view has a circular shape. The insertion part 34 is partially bent or rotated around an axis of the insertion part 34 by an operation of the operating part 32. As a result, the insertion part 34 is moved to a back side of the body while being bent according to a shape of the inside of the body (for example, a shape of the bronchus) or while being rotated around the axis of the insertion part 34 according to an internal part of the body.

A distal end part 36 of the insertion part 34 is provided with an endoscope 38, an illumination device 40, and a treatment tool opening 42. The endoscope 38 images the inside of the bronchus. Examples of the endoscope 38 include a CMOS camera. This is merely an example, and another type of camera such as a CCD camera may be used. The illumination device 40 irradiates the inside of the bronchus with light (for example, visible light). The treatment tool opening 42 is an opening through which a treatment tool 44 protrudes from the distal end part 36. The treatment tool 44 is inserted into the insertion part 34 through a treatment tool insertion opening 45. The treatment tool 44 passes through the insertion part 34 and protrudes into the bronchus through the treatment tool opening 42. In the example illustrated in FIG. 2, as the treatment tool 44, a puncture needle 44A protrudes through the treatment tool opening 42. It should be noted that, here, the puncture needle 44A has been described as an example of the treatment tool 44, but this is merely an example, and the treatment tool 44 may be, for example, grip forceps and/or a knife.

The endoscope apparatus 12 comprises a control device 46 and a light source device 48. The bronchoscope 18 is connected to the control device 46 and the light source device 48 through a cable 50. The control device 46 is a device that controls the entire endoscope apparatus 12. The light source device 48 is a device that emits light under the control of the control device 46 and supplies the light to the illumination device 40.

The control device 46 is provided with a plurality of hard keys 52. The plurality of hard keys 52 receive instructions from the user. A touch panel 54 is provided on the screen of the display device 14. The touch panel 54 is electrically connected to the control device 46 and receives the instruction from the user. The display device 14 is also electrically connected to the control device 46.

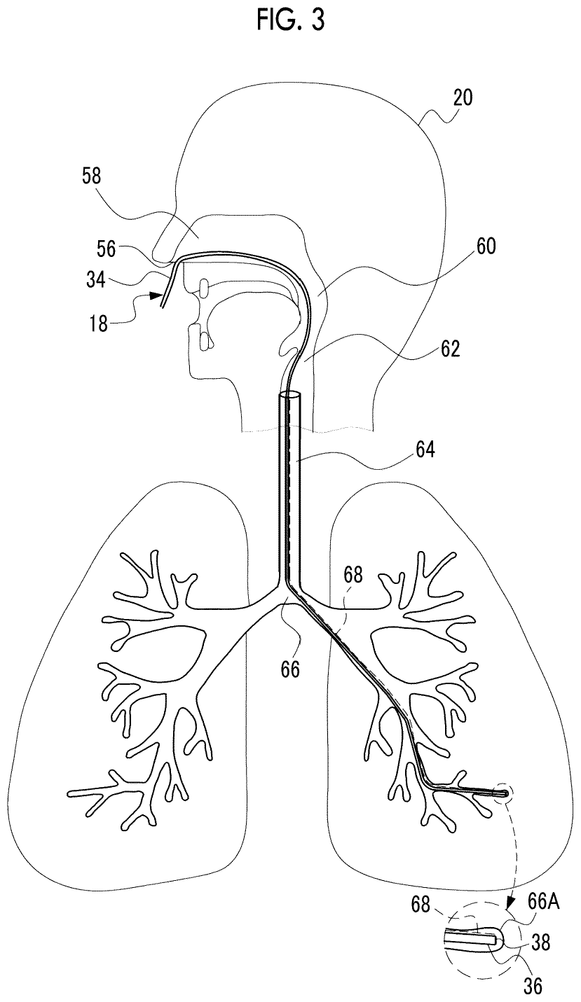

As an example, as illustrated in FIG. 3, the insertion part 34 of the bronchoscope 18 is inserted into a bronchus 66 from a nostril 56 of the subject 20 through a nasal cavity 58, a pharynx 60, a larynx 62, and a trachea 64. The distal end part 36 is moved to a back side of the bronchus 66 along a scheduled pathway 68 in the bronchus 66. The distal end part 36 moved to the back side of the bronchus 66 eventually reaches a target position 66A in the bronchus 66 (for example, a terminal of the bronchus 66). In a case where the distal end part 36 reaches the target position 66A, a treatment (for example, collection of a sample) is performed by the treatment tool 44 of the distal end part 36. During a period in which the distal end part 36 is inserted into the body of the subject 20, the endoscope 38 images the inside of the bronchus 66 at a predetermined frame rate. Examples of the predetermined frame rate include several tens of frames/second (for example, 30 frames/second or 60 frames/second).

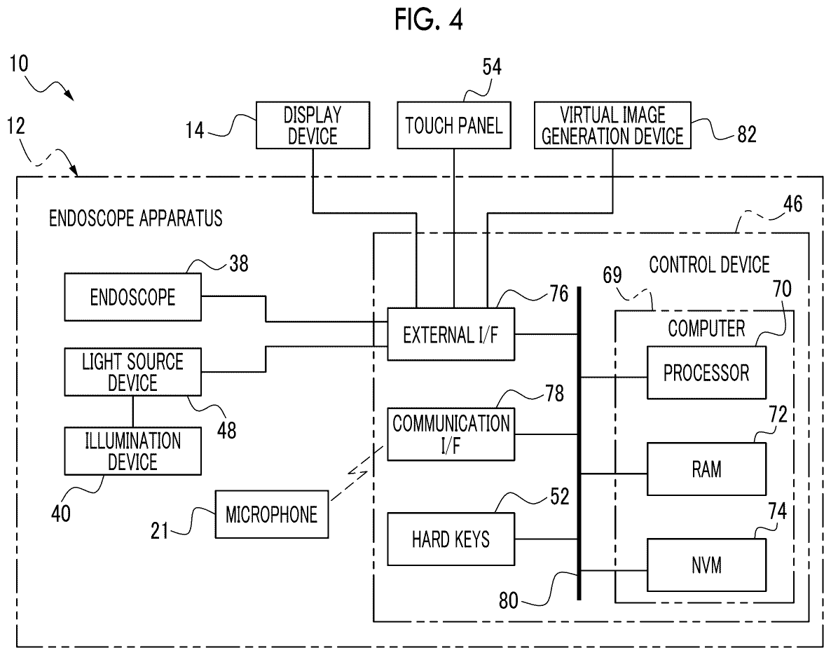

As an example, as illustrated in FIG. 4, the control device 46 comprises a computer 69. The computer 69 is an example of an “image processing apparatus” and a “computer” according to the technology of the present disclosure. The computer 69 comprises a processor 70, a RAM 72, and an NVM 74, and the processor 70, the RAM 72, and the NVM 74 are electrically connected to each other. The processor 70 is an example of a “processor” according to the technology of the present disclosure.

The control device 46 comprises the hard keys 52, an external I/F 76, and a communication I/F 78. The hard keys 52, the processor 70, the RAM 72, the NVM 74, the external I/F 76, and the communication I/F 78 are connected to a bus 80.

For example, the processor 70 includes a CPU and a GPU and controls the entire control device 46. The GPU is operated under the control of the CPU and is responsible for execution of various types of graphics-related processing. It should be noted that the processor 70 may be one or more CPUs with which the functions of the GPU are integrated or may be one or more CPUs with which the functions of the GPU are not integrated.

The RAM 72 is a memory that temporarily stores information, and is used as a work memory by the processor 70. The NVM 74 is a non-volatile storage device that stores various programs and various parameters.

Examples of the NVM 74 include a flash memory (for example, an EEPROM and/or an SSD). It should be noted that the flash memory is merely an example, and may be another non-volatile storage device, such as an HDD or a combination of two or more types of non-volatile storage devices.

The hard keys 52 receive the instructions from the user and output signals indicating the received instructions to the processor 70. Therefore, the instructions received by the hard keys 52 are recognized by the processor 70.

The external I/F 76 controls the exchange of various types of information between a device (hereinafter, also referred to as an “external device”) outside the control device 46 and the processor 70. Examples of the external I/F 76 include a USB interface.

The endoscope 38 as one of the external devices is connected to the external I/F 76, and the external I/F 76 controls the exchange of various types of information between the endoscope 38 and the processor 70. The processor 70 controls the endoscope 38 via the external I/F 76. In addition, the processor 70 acquires, via the external I/F 76, the endoscopic image 28 (see FIG. 1) obtained by imaging the inside of the bronchus 66 via the endoscope 38.

The light source device 48 as one of the external devices is connected to the external I/F 76, and the external I/F 76 controls the exchange of various types of information between the light source device 48 and the processor 70. The light source device 48 supplies the light to the illumination device 40 under the control of the processor 70. The illumination device 40 applies the light supplied from the light source device 48.

The display device 14 as one of the external devices is connected to the external I/F 76, and the processor 70 controls the display device 14 via the external I/F 76 so that the display device 14 displays various types of information.

The touch panel 54 as one of the external devices is connected to the external I/F 76, and the processor 70 acquires the instruction received by the touch panel 54 via the external I/F 76.

A virtual image generation device 82 as one of the external devices is connected to the external I/F 76. Examples of the virtual image generation device 82 include a server. It should be noted that the server is merely an example, and the virtual image generation device 82 may be a personal computer. The virtual image generation device 82 generates the virtual image 30 (see FIG. 1). The external I/F 76 controls the exchange of various types of information between the virtual image generation device 82 and the processor 70. The processor 70 requests the virtual image generation device 82 to provide a service (for example, to generate the virtual image 30 and provide the generated virtual image 30) via the external I/F 76, or acquires the virtual image 30 from the virtual image generation device 82 via the external I/F 76.

The communication I/F 78 is an interface including, for example, an antenna and a communication processor. For example, the communication I/F 78 performs wireless communication with a communication device using a communication system such as Wi-Fi (registered trademark) or Bluetooth (registered trademark), to control the exchange of various types of information between the communication device and the processor 70. Examples of the communication device include the microphone 21. The processor 70 acquires the voice signal from the microphone 21 via the communication I/F 78.

Meanwhile, the endoscopic image 28 (see FIG. 1) is displayed as the live view image on the display device 14 (see FIG. 1). The doctor 16 (see FIG. 1) operates the bronchoscope 18 while visually recognizing the endoscopic image 28 displayed on the display device 14, to cause the distal end part 36 (see FIG. 3) of the bronchoscope 18 to reach the target position 66A (see FIG. 3) in the bronchus 66. In this case, the doctor 16 moves the distal end part 36 of the bronchoscope 18 along the pathway 68 (see FIG. 3) in the bronchus. The pathway 68 is determined in advance. Each time a bifurcation appears in the bronchus 66 shown by the endoscopic image 28, the doctor 16 needs to advance the distal end part 36 toward the target position 66A while selecting a correct-direction bronchus 66.

In order to guide the doctor 16 to the correct-direction bronchus 66, the display device 14 displays the virtual image 30 (see FIG. 1) as a reference moving image in a state of being arranged side by side with the endoscopic image 28. The virtual image 30 is a moving image prepared in advance as a moving image showing an aspect in which the inside of the bronchus 66 is observed along the pathway 68. Therefore, the doctor 16 can operate the bronchoscope 18 while comparing the virtual image 30 with the endoscopic image 28 to select the correct-direction bronchus 66 and advance the distal end part 36 toward the target position 66A.

However, a display aspect of the virtual image 30 displayed on the display device 14 may not be a display aspect desired by the doctor 16. For example, in a case where a speed at which the display of the virtual image 30 displayed on the display device 14 is advanced is not a speed desired by the doctor 16, the work of inserting the bronchoscope 18 may be advanced at a pace not desired by the doctor 16. For example, a case where a portion shown by the virtual image 30 has not caught up with a portion shown by the endoscopic image 28 may be one of the causes for the doctor 16 to wait until the display of the virtual image 30 catches up with the portion shown by the endoscopic image 28. On the contrary, a case where the portion shown by the virtual image 30 has advanced beyond the portion shown by the endoscopic image 28 may be one of the causes of rushing the work of the doctor 16.

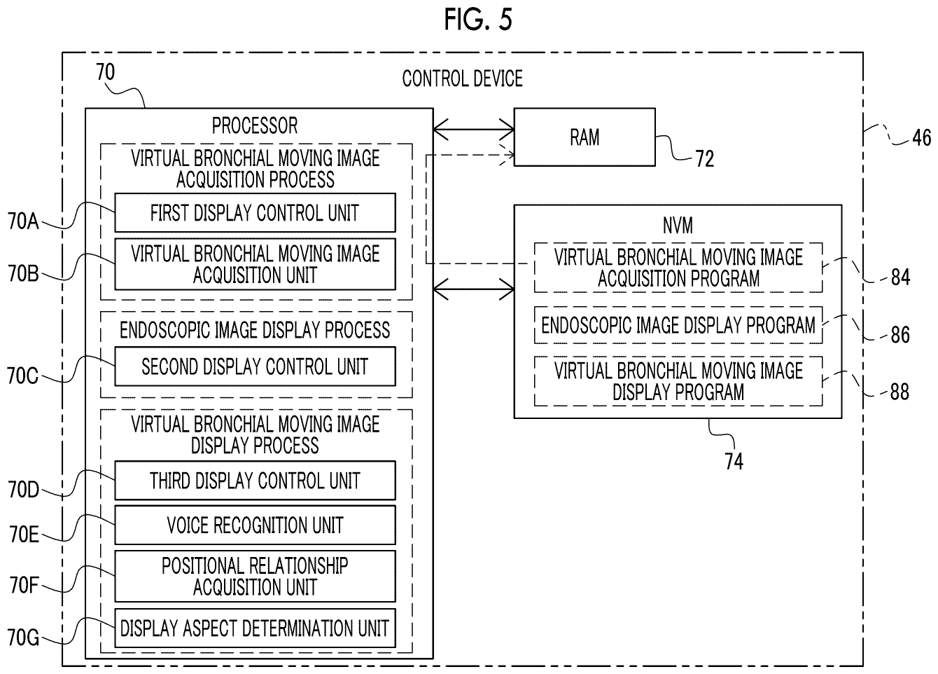

Therefore, in view of such circumstances, in the present embodiment, as illustrated in FIG. 5 as an example, the processor 70 performs a virtual bronchial moving image acquisition process, an endoscopic image display process, and a virtual bronchial moving image display process. The NVM 74 stores a virtual bronchial moving image acquisition program 84, an endoscopic image display program 86, and a virtual bronchial moving image display program 88.

The processor 70 reads out the virtual bronchial moving image acquisition program 84 from the NVM 74 and executes the read out virtual bronchial moving image acquisition program 84 on the RAM 72 to perform the virtual bronchial moving image acquisition process. The virtual bronchial moving image acquisition process is implemented by the processor 70 operating as a first display control unit 70A and a virtual bronchial moving image acquisition unit 70B according to the virtual bronchial moving image acquisition program 84.

The processor 70 reads out the endoscopic image display program 86 from the NVM 74 and executes the read out endoscopic image display program 86 on the RAM 72 to perform the endoscopic image display process. The endoscopic image display process is implemented by the processor 70 operating as a second display control unit 70C according to the endoscopic image display program 86.

The processor 70 reads out the virtual bronchial moving image display program 88 from the NVM 74 and executes the read out virtual bronchial moving image display program 88 on the RAM 72 to perform the virtual bronchial moving image display process. The virtual bronchial moving image display process is implemented by the processor 70 operating as a third display control unit 70D, a voice recognition unit 70E, a positional relationship acquisition unit 70F, and a display aspect determination unit 70G according to the virtual bronchial moving image display program 88. The virtual bronchial moving image display program 88 is an example of a “program” according to the technology of the present disclosure.

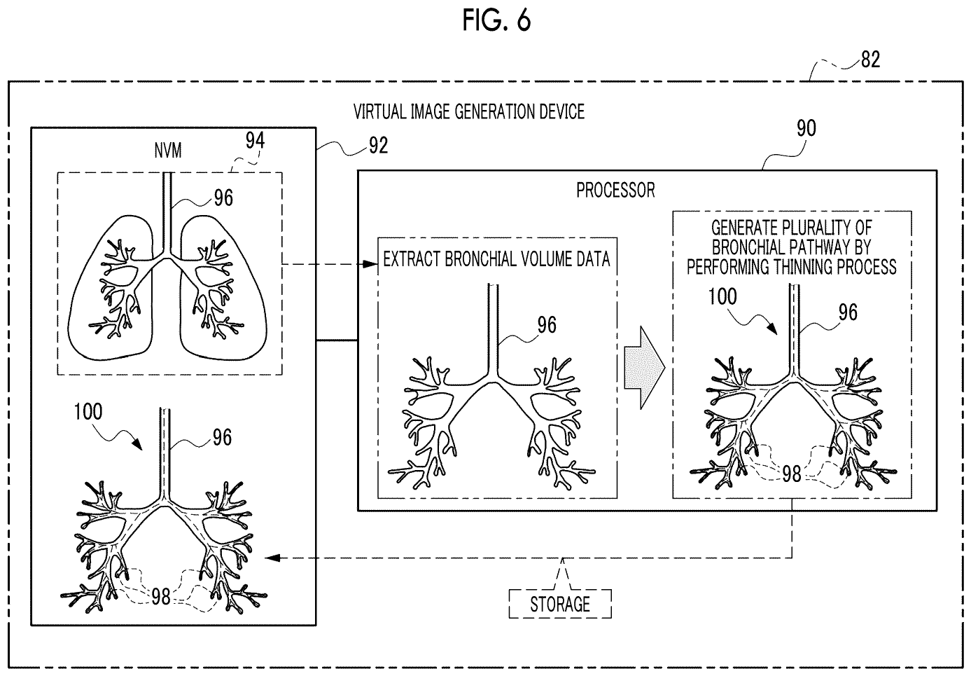

As an example, as illustrated in FIG. 6, the virtual image generation device 82 comprises a processor 90, an NVM 92, and a RAM (not illustrated), and the processor 90 executes a virtual image generation program (not illustrated) on the RAM to perform a virtual image generation process. Hereinafter, examples of the contents of the virtual image generation process performed by the processor 90 and the contents of the virtual bronchial moving image acquisition process performed by the processor 70 of the control device 46 will be described with reference to FIGS. 6 to 8.

In the virtual image generation device 82, volume data 94 is stored in the NVM 92. The volume data 94 is an example of “volume data” according to the technology of the present disclosure. The volume data 94 is a three-dimensional image obtained by stacking a plurality of two-dimensional slice images obtained by imaging the whole body or a part of the body (for example, a chest) of the subject 20 depending on the modality and dividing the stacked images into voxels. A position of each voxel is specified by three-dimensional coordinates. Examples of the modality include a CT apparatus. The CT apparatus is merely an example, and other examples of the modality include an MRI apparatus and an ultrasound diagnostic apparatus.

The volume data 94 includes bronchial volume data 96 which is a three-dimensional image showing the trachea 64 and the bronchus 66 of the subject 20. The bronchial volume data 96 is an example of a “bronchial image” according to the technology of the present disclosure.

The processor 90 extracts the bronchial volume data 96 from the volume data 94. Then, the processor 90 performs a thinning process on the bronchial volume data 96 to generate a plurality of bronchial pathways 98. The bronchial pathway 98 is a three-dimensional line passing through the center of the virtual bronchus (hereinafter, also referred to as a “virtual bronchus”) shown by the bronchial volume data 96 in a cross-sectional view. The three-dimensional line passing through the center of the virtual bronchus in a cross-sectional view is obtained by thinning the bronchial volume data 96. The number of bronchial pathways 98 corresponds to the number of terminals of the bronchus shown by the bronchial volume data 96.

The processor 90 stores pathway-inclusive bronchial volume data 100 in the NVM 92. The pathway-inclusive bronchial volume data 100 is a three-dimensional image obtained by integrating the bronchial volume data 96 and the bronchial pathway 98.

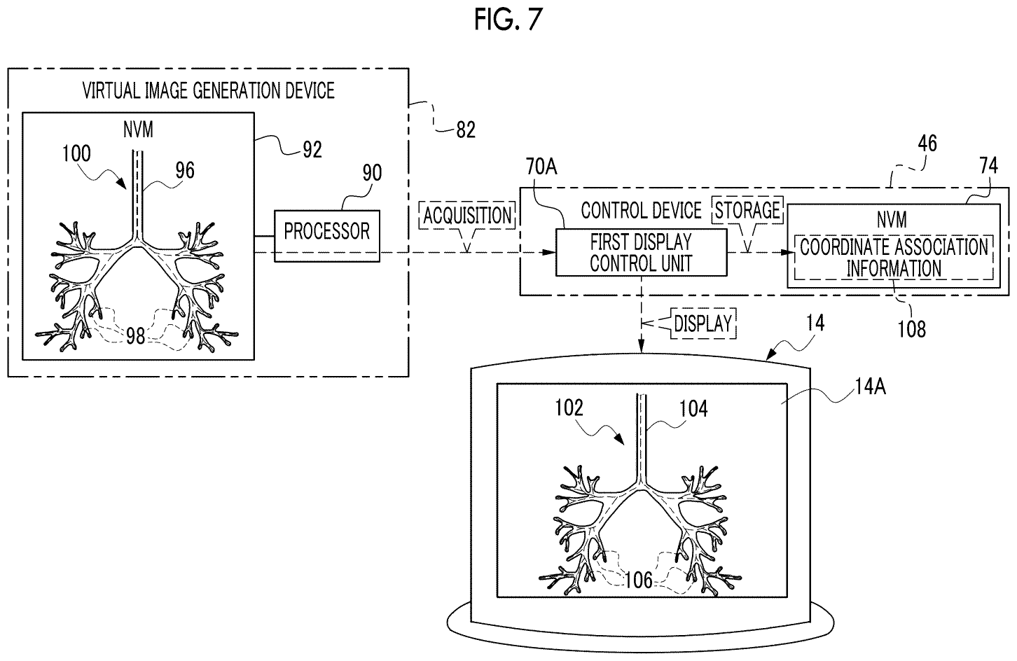

As illustrated in FIG. 7 as an example, in the control device 46, the first display control unit 70A acquires the pathway-inclusive bronchial volume data 100 from the NVM 92 of the virtual image generation device 82 via the processor 90. Then, the first display control unit 70A displays a pathway-inclusive bronchial image 102 on the display device 14. The pathway-inclusive bronchial image 102 is generated by the first display control unit 70A based on the pathway-inclusive bronchial volume data 100. The pathway-inclusive bronchial image 102 is an image in which the pathway-inclusive bronchial volume data 100 is rendered on a screen 14A of the display device 14. The pathway-inclusive bronchial image 102 is a rendering image obtained by integrating a bronchial image 104 and a bronchial pathway 106. The bronchial image 104 is a rendering image corresponding to the bronchial volume data 96, and the bronchial pathway 106 is a rendering image corresponding to the bronchial pathway 98.

The first display control unit 70A stores coordinate association information 108 in the NVM 74. The coordinate association information 108 is information in which the three-dimensional coordinates before the rendering (that is, the three-dimensional coordinates of the pathway-inclusive bronchial volume data 100) and the two-dimensional coordinates after the rendering (that is, the two-dimensional coordinates of the pathway-inclusive bronchial image 102) are associated with each other.

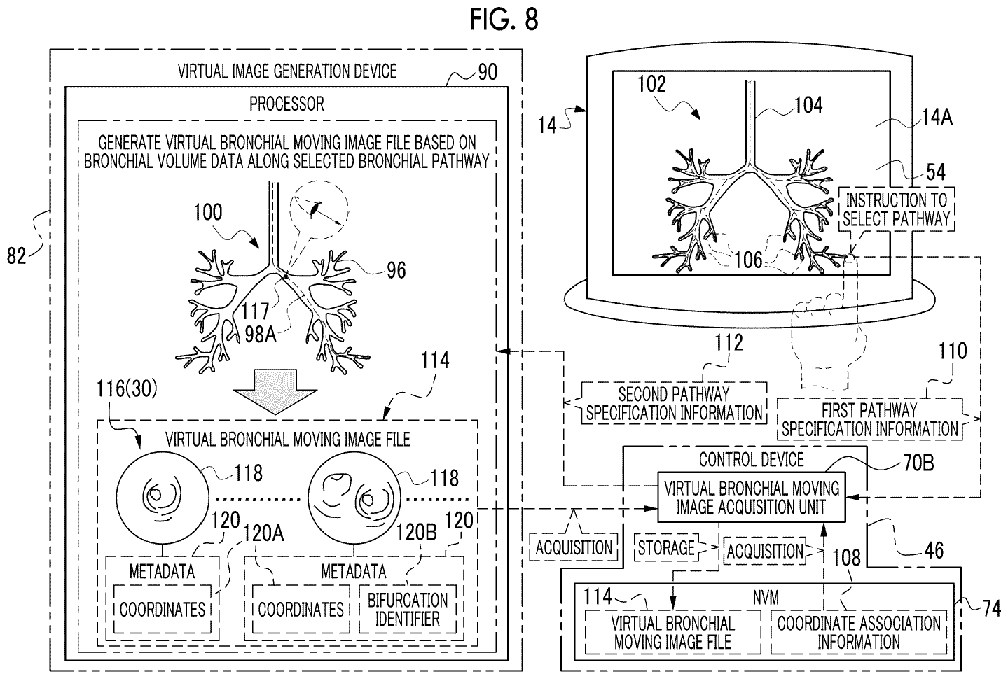

As illustrated in FIG. 8 as an example, in a state where the pathway-inclusive bronchial image 102 is displayed on the screen 14A of the display device 14, the touch panel 54 receives a pathway selection instruction from the user. In the example illustrated in FIG. 8, an aspect is illustrated in which the pathway selection instruction is given to the touch panel 54 by the user's finger. The pathway selection instruction is an instruction to select one bronchial pathway 106 from among a plurality of bronchial pathways 106.

For example, in a case where an instruction to select end point coordinates as coordinates for specifying an end point among the plurality of bronchial pathways 106 is received by the touch panel 54, the bronchial pathway 106 including the end point specified by the selected end point coordinates is selected.

The virtual bronchial moving image acquisition unit 70B acquires first pathway specification information 110 from the touch panel 54. For example, the first pathway specification information 110 is the end point coordinates selected by the user via the touch panel 54. The virtual bronchial moving image acquisition unit 70B acquires the coordinate association information 108 from the NVM 74, and converts the first pathway specification information 110 into second pathway specification information 112 with reference to the coordinate association information 108. The conversion of the first pathway specification information 110 into the second pathway specification information 112 is implemented by acquiring the three-dimensional coordinates corresponding to the first pathway specification information 110 (here, as an example, the end point coordinates) from the coordinate association information 108 as the second pathway specification information 112. The virtual bronchial moving image acquisition unit 70B outputs the second pathway specification information 112 to the processor 90 of the virtual image generation device 82.

In the virtual image generation device 82, the processor 90 refers to the second pathway specification information 112 input from the virtual bronchial moving image acquisition unit 70B, to select a bronchial pathway 98A, which is one bronchial pathway 98, from among the plurality of bronchial pathways 98. Here, the bronchial pathway 98A is the bronchial pathway 98 including the second pathway specification information 112 (here, as an example, the three-dimensional coordinates corresponding to the end point coordinates selected by the user) among the plurality of bronchial pathways 98.

The processor 90 generates a virtual bronchial moving image file 114 based on the bronchial volume data 96 along the bronchial pathway 98A. The bronchial volume data 96 along the bronchial pathway 98A means the bronchial volume data 96 showing the trachea 64 and the bronchus 66 through which the bronchial pathway 98A passes (that is, a portion of the bronchial volume data 96 in which the thinning process for generating the bronchial pathway 98A is performed).

The virtual bronchial moving image file 114 includes a virtual bronchial moving image 116. The virtual bronchial moving image 116 is an example of the virtual image 30 illustrated in FIG. 1. The virtual bronchial moving image 116 is a moving image showing an aspect in which a back side of the inside of the virtual bronchus is observed from a viewpoint 117 set on the bronchial pathway 98A in the virtual bronchus shown by the bronchial volume data 96 (that is, in a terminal direction of the bronchial pathway 98A).

The virtual bronchial moving image 116 includes a plurality of frames 118 obtained from a start point to an end point of the bronchial pathway 98A according to a predetermined frame rate. The plurality of frames 118 are arranged in time series. Metadata 120 is associated with each frame 118. The metadata 120 includes coordinates 120A. The coordinates 120A are three-dimensional coordinates of a position at which the corresponding frame 118 (that is, the frame 118 associated with the metadata 120) is obtained among a plurality of three-dimensional coordinates included in the bronchial pathway 98A. In addition, among a plurality of pieces of metadata 120, the metadata 120 related to the frame 118 at the position corresponding to the bifurcation in the virtual bronchus includes a bifurcation identifier 120B that is an identifier for specifying the bifurcation in the virtual bronchus.

In a case where the virtual bronchial moving image file 114 is generated by the processor 90 of the virtual image generation device 82, the virtual bronchial moving image acquisition unit 70B acquires the virtual bronchial moving image file 114 from the virtual image generation device 82. Then, the virtual bronchial moving image acquisition unit 70B stores the virtual bronchial moving image file 114 acquired from the virtual image generation device 82 in the NVM 74.

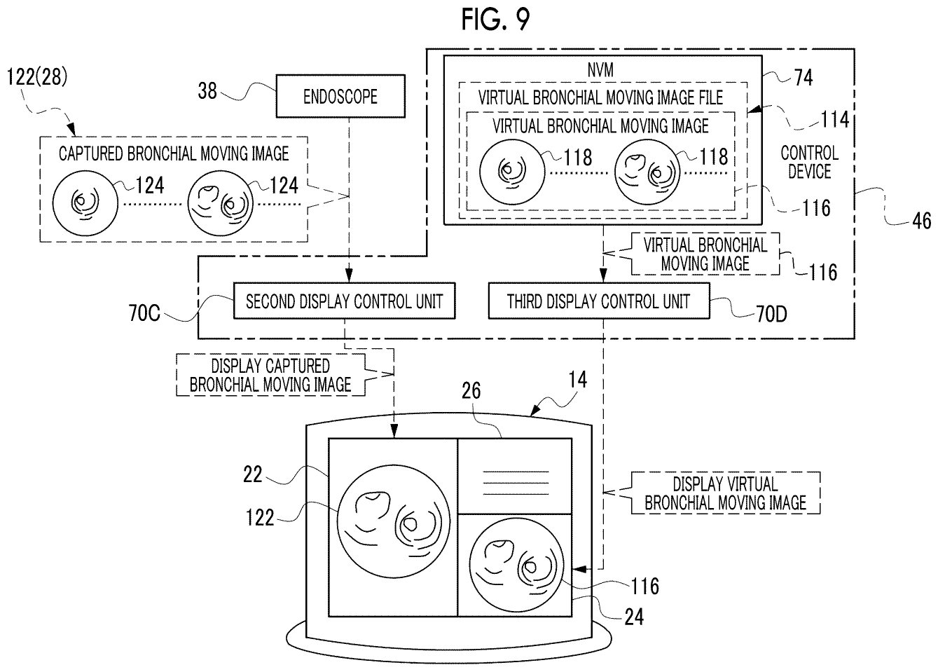

As an example, as illustrated in FIG. 9, in the control device 46, the second display control unit 70C acquires a captured bronchial moving image 122 from the endoscope 38. The captured bronchial moving image 122 is an example of the endoscopic image 28 illustrated in FIG. 1. The captured bronchial moving image 122 is a moving image (here, for example, a live view image) obtained by imaging an inside of the trachea 64 and the inside of the bronchus 66 (see FIG. 3) along the pathway 68 (see FIG. 3) via the endoscope 38. The captured bronchial moving image 122 includes a plurality of frames 124 obtained by performing imaging according to a predetermined frame rate from a start point to an end point of the pathway 68. The second display control unit 70C outputs the plurality of frames 124 to the display device 14 in time series, to display the captured bronchial moving image 122 on the first screen 22 of the display device 14.

In the control device 46, the third display control unit 70D acquires the virtual bronchial moving image 116 from the NVM 74. Then, the third display control unit 70D outputs the plurality of frames 118 to the display device 14 in time series, to display the virtual bronchial moving image 116 on the second screen 24 of the display device 14. It should be noted that examples of a trigger for starting the display of the virtual bronchial moving image 116 (that is, a trigger for the third display control unit 70D to start outputting the virtual bronchial moving image 116) on the display device 14 include a trigger that a start instruction (that is, an instruction to start the display of the virtual bronchial moving image 116) from the user is received by a reception device (hereinafter, also simply referred to as the “reception device”) such as the microphone 21, the touch panel 54, or the hard keys 52.

A speed at which the display of the virtual bronchial moving image 116 is advanced is essentially a fixed speed unless an instruction from the user (for example, an instruction performed by a voice of the doctor 16) is given to the control device 46. Examples of the fixed speed include a speed calculated from a distance from the start point to the end point of the bronchial pathway 98A and a default time required for the viewpoint 117 to move from the start point to the end point of the bronchial pathway 98A.

The display aspect including the speed at which the display of the virtual bronchial moving image 116 is advanced is changed on the condition that the instruction from the user (for example, an instruction performed by the voice of the doctor 16) is given to the control device 46, and in a case where the instruction from the user is released, the display aspect is restored to a default display aspect. The instruction given to the control device 46 is received by the reception device. For example, the speed at which the display of the virtual bronchial moving image 116 is advanced is changed according to the instruction received by the reception device. The change in the speed at which the display of the virtual bronchial moving image 116 is advanced is implemented by so-called fast forwarding, frame advance, slow play, or the like.

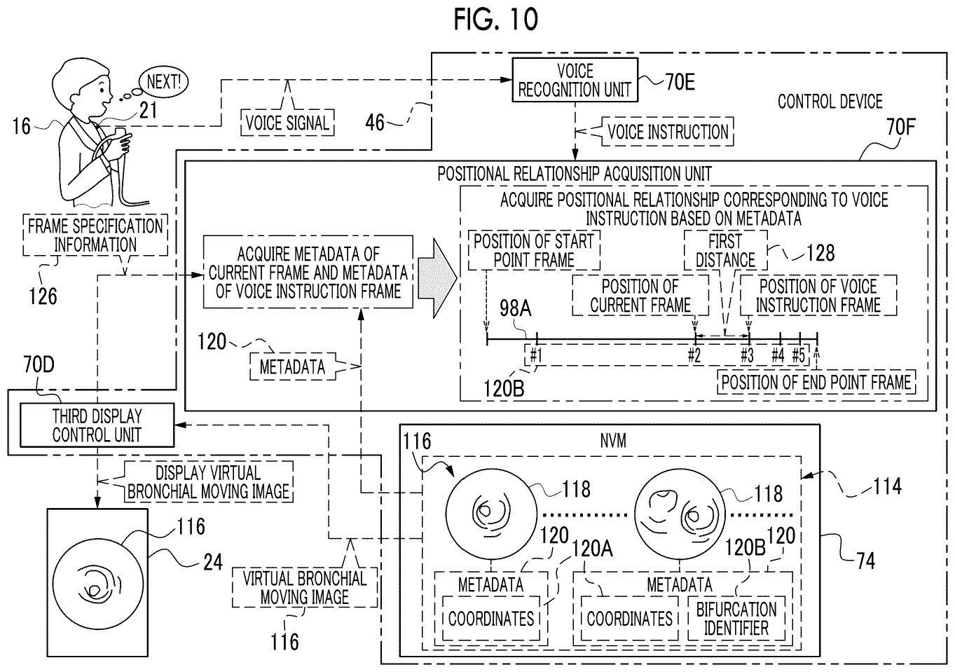

As an example, as illustrated in FIG. 10, the microphone 21 outputs the voice uttered by the doctor 16 as the voice signal to the voice recognition unit 70E. The voice recognition unit 70E recognizes the voice indicated by the voice signal input from the microphone 21. The voice recognition is implemented using a known technology. In a case where the voice recognition unit 70E recognizes a voice instruction, which is an instruction performed by the voice of the doctor 16, the positional relationship acquisition unit 70F receives the voice instruction output by the voice recognition unit 70E. The voice instruction includes an instruction related to a position to which the viewpoint 117 is moved on the bronchial pathway 98A. For example, a voice instruction “next” uttered by the doctor 16 is an instruction to move the viewpoint 117 to the position of the bifurcation to be displayed next as the frame 118 on the bronchial pathway 98A. It should be noted that, here, for convenience of description, the voice instruction “next” has been described as an example, but this is merely an example, and any voice instruction may be used as long as the position on the bronchial pathway 98 can be specified.

The positional relationship acquisition unit 70F acquires a positional relationship between a plurality of positions from an upstream side to a downstream side in the virtual bronchus according to the voice instruction. Here, for example, the positional relationship between the plurality of positions is defined by a distance between the plurality of positions. That is, the positional relationship acquisition unit 70F acquires a first distance 128, which is a distance between the plurality of positions from the upstream side to the downstream side in the virtual bronchus, according to the voice instruction. The first distance 128 is an example of a “first distance” according to the technology of the present disclosure.

In the example illustrated in FIG. 10, an aspect is illustrated in which the virtual bronchial moving image 116 is displayed on the second screen 24 by the third display control unit 70D. Hereinafter, for the purpose of facilitating the understanding of the technology of the present disclosure, an example of a case where the voice instruction “next” from the doctor 16 is given to the control device 46 in a state where the virtual bronchial moving image 116 is displayed on the second screen 24 will be described.

In a case where the voice instruction is received, the positional relationship acquisition unit 70F acquires frame specification information 126 from the third display control unit 70D. The frame specification information 126 is information for specifying the frame 118 displayed on the second screen 24 at the current point in time (for example, a number for specifying the frame 118 or a time stamp indicating a time at which the frame 118 is obtained). The positional relationship acquisition unit 70F acquires the metadata 120 of the frame 118 (hereinafter, also referred to as a “current frame”) specified from the frame specification information 126. In addition, the positional relationship acquisition unit 70F acquires the metadata 120 of the frame 118 (hereinafter, also referred to as a “voice instruction frame”) specified from the voice instruction with reference to the bifurcation identifier 120B included in the metadata 120. That is, the metadata 120 including the bifurcation identifier 120B related to the bifurcation to be displayed next as an image included in the frame 118 on the second screen 24 is acquired by the positional relationship acquisition unit 70F.

The positional relationship acquisition unit 70F calculates the first distance 128 based on the coordinates 120A included in the metadata 120 of the current frame and the coordinates 120A included in the metadata 120 of the voice instruction frame. The first distance 128 is a distance from the coordinates 120A included in the metadata 120 of the current frame to the coordinates 120A included in the metadata 120 of the voice instruction frame.

For example, in a case where a position corresponding to the current frame on the bronchial pathway 98 and a position corresponding to the voice instruction frame are a position of an upstream bifurcation among a plurality of bifurcations included in the virtual bronchus (hereinafter, also referred to as an “upstream bifurcation”) and a position of a downstream bifurcation in the virtual bronchus (hereinafter, also referred to as a “downstream bifurcation”), the first distance 128 is a distance between the upstream bifurcation and the downstream bifurcation. It should be noted that the position of the upstream bifurcation is an example of a “position corresponding to the upstream bifurcation” according to the technology of the present disclosure, and the position of the downstream bifurcation is an example of a “position corresponding to the downstream bifurcation” according to the technology of the present disclosure.

In the example illustrated in FIG. 10, in a range from the position of the frame 118 corresponding to the start point of the bronchial pathway 98A to the position of the frame 118 corresponding to the end point of the bronchial pathway 98A, a distance between the upstream bifurcation to which “#2” is assigned as the bifurcation identifier 120B and the downstream bifurcation to which “#3” is assigned as the bifurcation identifier 120B is illustrated as the first distance 128.

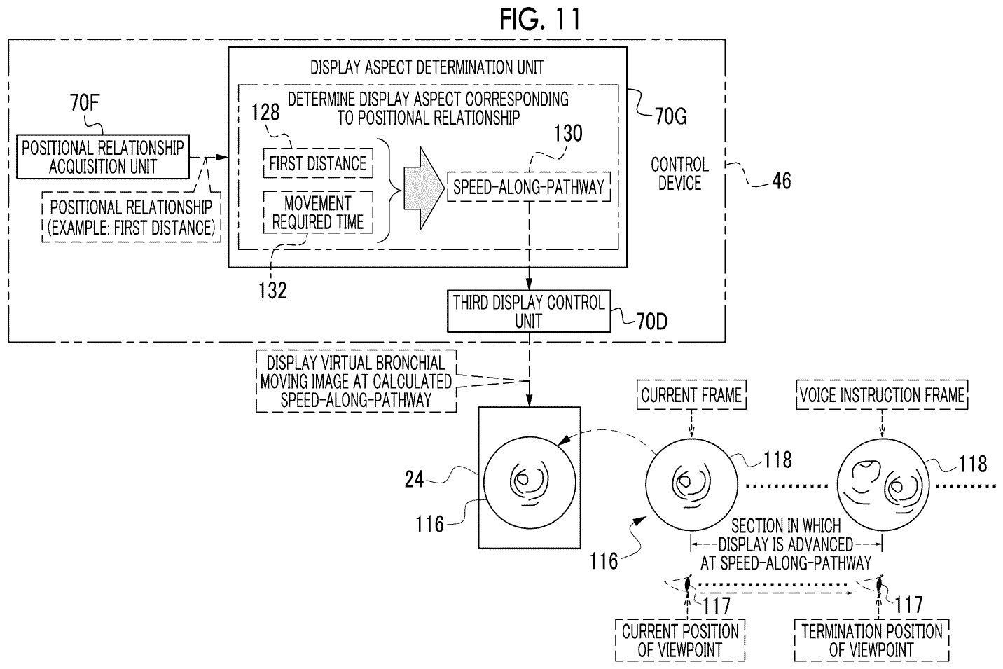

As illustrated in FIG. 11 as an example, the display aspect determination unit 70G determines a display aspect corresponding to the positional relationship acquired by the positional relationship acquisition unit 70F. Here, the display aspect means a display aspect of the virtual bronchial moving image 116.

Here, as the display aspect determined by the display aspect determination unit 70G, an aspect in which the display is advanced at a speed-along-pathway 130 will be described as an example. The speed-along-pathway 130 means a speed at which the display of the virtual bronchial moving image 116 is advanced along the bronchial pathway 98. The speed-along-pathway 130 is a speed determined according to the first distance 128 calculated by the positional relationship acquisition unit 70F as the positional relationship between the plurality of positions from the upstream side to the downstream side in the virtual bronchus, and a movement required time 132. That is, the display aspect determination unit 70G calculates the speed-along-pathway 130 (for example, the first distance 128/the movement required time 132) based on the first distance 128 calculated by the positional relationship acquisition unit 70F and the movement required time 132. The speed-along-pathway 130 is a fixed speed (that is, a constant speed). The movement required time 132 means a time required for the viewpoint 117 to move between the plurality of positions from the upstream side to the downstream side in the virtual bronchus (that is, a time required from the display of the current frame to the display of the voice instruction frame).

The movement required time 132 may be determined for each relationship between a current position of the viewpoint 117 and a movement destination (a position specified by the voice instruction) of the viewpoint 117, may be determined according to the instruction received by the reception device, or may be determined according to an actual insertion amount of the insertion part 34 of the bronchoscope 18 (hereinafter, also referred to as an “actual insertion amount”).

In a case where the movement required time 132 is determined by using the insertion amount, for example, ideal insertion amount data indicating an ideal insertion amount may be included in the metadata 120 of the frame 118 in advance, and the movement required time 132 may be determined according to a difference between an ideal insertion amount indicated by the ideal insertion amount data and the actual insertion amount. For example, in a case where the actual insertion amount is larger than the ideal insertion amount, the display aspect determination unit 70G determines that the display of the virtual bronchial moving image 116 has not been advanced as much as the assumption of the doctor 16, and makes the movement required time 132 shorter as the actual insertion amount is larger. On the contrary, in a case where the actual insertion amount is smaller than the ideal insertion amount, the display aspect determination unit 70G determines that the display of the virtual bronchial moving image 116 has been advanced as much as the assumption of the doctor 16, and makes the movement required time 132 longer as the actual insertion amount is smaller.

In addition, the display aspect determination unit 70G may adjust the speed-along-pathway 130 according to the position of the current frame (that is, the current position of the viewpoint 117). For example, the speed-along-pathway 130 may be increased as the position of the current frame (that is, the current position of the viewpoint 117) is closer to the start point of the bronchial pathway 98A, or the speed-along-pathway 130 may be adjusted by using different coefficients for each bifurcation on the bronchial pathway 98A.

The third display control unit 70D displays the virtual bronchial moving image 116 in the display aspect determined by the display aspect determination unit 70G. Here, as an example, the third display control unit 70D displays the virtual bronchial moving image 116 on the second screen 24 at the speed-along-pathway 130 calculated by the display aspect determination unit 70G. That is, the display of the virtual bronchial moving image 116 is advanced at the speed-along-pathway 130 in a section from the current frame (that is, the current position of the viewpoint 117) in the virtual bronchial moving image 116 to the voice instruction frame (that is, a termination position indicated as the movement destination of the viewpoint 117 by the doctor 16). It should be noted that, in the following description, the termination position indicated as the movement destination of the viewpoint 117 by the doctor 16 will be simply referred to as a “termination position”.

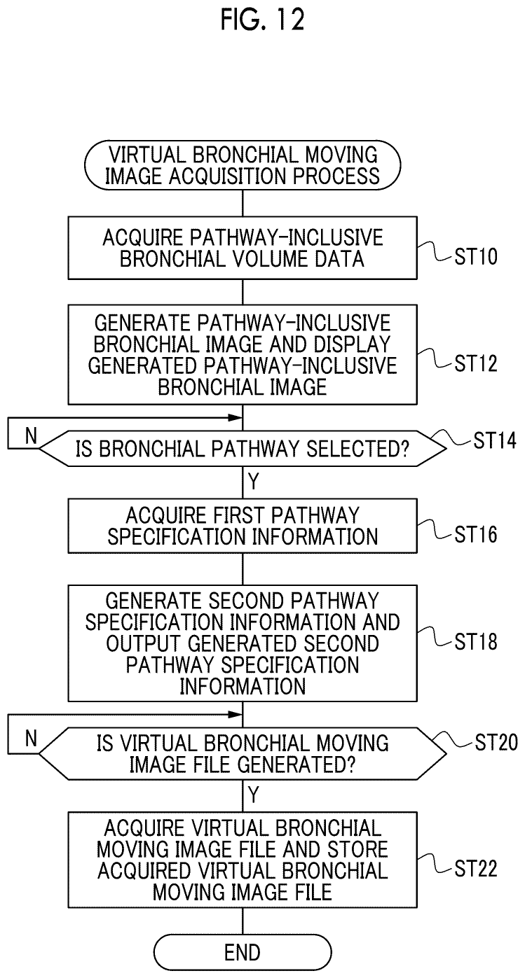

Hereinafter, the actions of the endoscope system 10 will be described with reference to FIGS. 12 to 14.

First, an example of a flow of the virtual bronchial moving image acquisition process performed by the processor 70 of the control device 46 in a case where an instruction to start the execution of the virtual bronchial moving image acquisition process is received by the reception device will be described with reference to FIG. 12.

In the virtual bronchial moving image acquisition process illustrated in FIG. 12, first, in step ST10, the first display control unit 70A acquires the pathway-inclusive bronchial volume data 100 (see FIG. 7). After the process of step ST10 is executed, the virtual bronchial moving image acquisition process proceeds to step ST12.

In step ST12, the first display control unit 70A generates the pathway-inclusive bronchial image 102 based on the pathway-inclusive bronchial volume data 100 acquired in step ST10, and displays the generated pathway-inclusive bronchial image 102 on the screen 14A (see FIG. 7). After the process of step ST12 is executed, the virtual bronchial moving image acquisition process proceeds to step ST14.

In step ST14, the virtual bronchial moving image acquisition unit 70B determines whether or not one bronchial pathway 106 is selected from among the plurality of bronchial pathways 106 included in the pathway-inclusive bronchial image 102 displayed on the screen 14A. In this case, for example, in a case where the pathway selection instruction is received by the touch panel 54 (see FIG. 8), it is determined that one bronchial pathway 106 is selected from among the plurality of bronchial pathways 106. In step ST14, in a case where one bronchial pathway 106 is not selected from among the plurality of bronchial pathways 106, a negative determination is made, and the determination in step ST14 is performed again. In step ST14, in a case where one bronchial pathway 106 is selected from among the plurality of bronchial pathways 106, an affirmative determination is made, and the virtual bronchial moving image acquisition process proceeds to step ST16.

In step ST16, the virtual bronchial moving image acquisition unit 70B acquires the first pathway specification information 110 from the touch panel 54 (see FIG. 8). After the process of step ST16 is executed, the virtual bronchial moving image acquisition process proceeds to step ST18.

In step ST18, the virtual bronchial moving image acquisition unit 70B generates the second pathway specification information 112 based on the first pathway specification information 110 acquired in step ST16. That is, the virtual bronchial moving image acquisition unit 70B converts the first pathway specification information 110 acquired in step ST16 into the second pathway specification information 112 with reference to the coordinate association information 108. Then, the virtual bronchial moving image acquisition unit 70B outputs the second pathway specification information 112 to the processor 90 of the virtual image generation device 82 (see FIG. 8). After the process of step ST18 is executed, the virtual bronchial moving image acquisition process proceeds to step ST20.

The processor 90 of the virtual image generation device 82 selects the bronchial pathway 98A (see FIG. 8) from among the plurality of bronchial pathways 98 included in the pathway-inclusive bronchial volume data 100 according to the second pathway specification information 112 input from the virtual bronchial moving image acquisition unit 70B. Then, the processor 90 generates the virtual bronchial moving image file 114 (see FIG. 8) based on the bronchial volume data 96 along the bronchial pathway 98A.

In step ST20, the virtual bronchial moving image acquisition unit 70B determines whether or not the virtual bronchial moving image file 114 is generated. In step ST20, in a case where the virtual bronchial moving image file 114 is not generated, a negative determination is made, and the determination in step ST20 is performed again. In step ST20, in a case where the virtual bronchial moving image file 114 is generated, an affirmative determination is made, and the virtual bronchial moving image acquisition process proceeds to step ST22.

In step ST22, the virtual bronchial moving image acquisition unit 70B acquires the virtual bronchial moving image file 114 from the virtual image generation device 82, and stores the acquired virtual bronchial moving image file 114 in the NVM 74 (see FIG. 8). After the process of step ST22 is executed, the virtual bronchial moving image acquisition process ends.

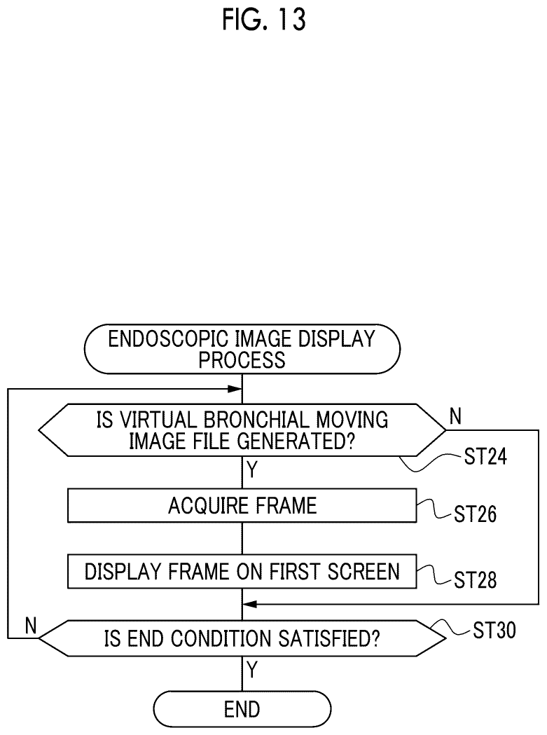

Next, an example of a flow of the endoscopic image display process performed by the processor 70 of the control device 46 in a case where the endoscope 38 (see FIG. 3) is inserted into the body of the subject 20 (for example, the trachea 64) will be described with reference to FIG. 13. It should be noted that, here, the description will be made on the premise that the endoscope 38 performs imaging at the predetermined frame rate along the pathway 68 (see FIG. 3) to acquire the captured bronchial moving image 122 (see FIG. 9) as the live view image.

In the endoscopic image display process illustrated in FIG. 13, first, in step ST24, the second display control unit 70C determines whether or not imaging for one frame is performed by the endoscope 38. In step ST24, in a case where the imaging for one frame is not performed by the endoscope 38, a negative determination is made, and the endoscopic image display process proceeds to step ST30. In step ST24, in a case where the imaging for one frame is performed by the endoscope 38, an affirmative determination is made, and the endoscopic image display process proceeds to step ST26.

In step ST26, the second display control unit 70C acquires the frame 124 obtained by performing the imaging for one frame via the endoscope 38 (see FIG. 9). After the process of step ST26 is executed, the endoscopic image display process proceeds to step ST28.

In step ST28, the second display control unit 70C displays the frame 124 acquired in step ST26 on the first screen 22 (see FIG. 9). After the process of step ST28 is executed, the endoscopic image display process proceeds to step ST30.

In step ST30, the second display control unit 70C determines whether or not a condition for ending the endoscopic image display process (hereinafter, referred to as an “endoscopic image display process end condition”) is satisfied. Examples of the endoscopic image display process end condition include a condition in which the reception device receives an instruction to end the endoscopic image display process. In step ST30, in a case where the endoscopic image display process end condition is not satisfied, a negative determination is made, and the endoscopic image display process proceeds to step ST24. In step ST30, in a case in which the endoscopic image display process end condition is satisfied, an affirmative determination is made, and the endoscopic image display process ends.

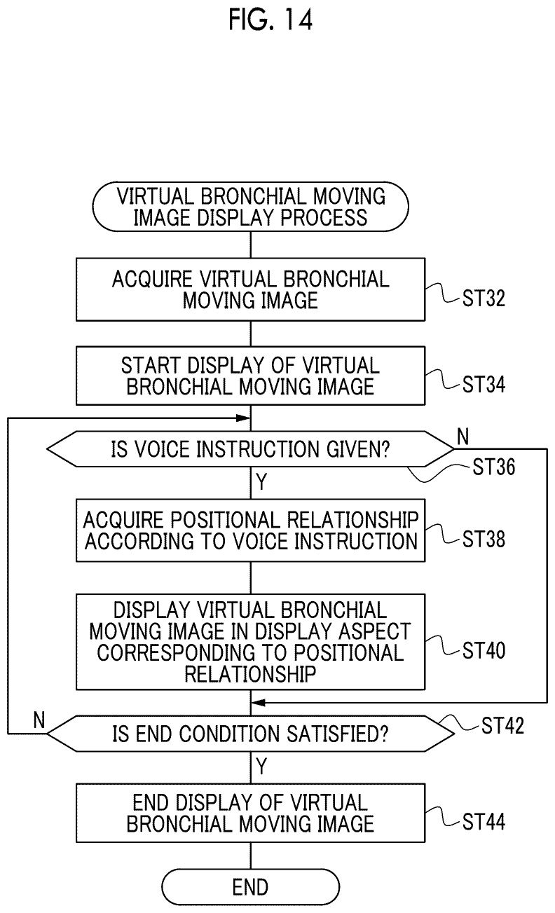

Next, an example of a flow of the virtual bronchial moving image display process performed by the processor 70 of the control device 46 in a case where an instruction to start the execution of the virtual bronchial moving image display process is received by the reception device will be described with reference to FIG. 14.

It should be noted that the flow of the virtual bronchial moving image display process illustrated in FIG. 14 is an example of an “image processing method” according to the technology of the present disclosure. In addition, here, the description is made on the premise that the virtual bronchial moving image file 114 is stored in the NVM 74.

In the virtual bronchial moving image display process illustrated in FIG. 14, first, in step ST32, the third display control unit 70D acquires the virtual bronchial moving image 116 from the NVM 74 (see FIG. 10). After the process of step ST32 is executed, the virtual bronchial moving image display process proceeds to step ST34.

In step ST34, the third display control unit 70D starts the display of the virtual bronchial moving image 116 acquired in step ST32 on the second screen 24 (see FIG. 10). After the process of step ST34 is executed, the virtual bronchial moving image display process proceeds to step ST36.

In step ST36, the positional relationship acquisition unit 70F determines whether or not the voice instruction is given from the doctor 16. In step ST36, in a case where the voice instruction is not given from the doctor 16, a negative determination is made, and the virtual bronchial moving image display process proceeds to step ST42. In step ST36, in a case where the voice instruction is given from the doctor 16, an affirmative determination is made, and the virtual bronchial moving image display process proceeds to step ST38.

In step ST38, the positional relationship acquisition unit 70F acquires the positional relationship between the plurality of positions from the upstream side to the downstream side in the virtual bronchus according to the voice instruction given by the doctor 16 (see FIG. 10). For example, here, the first distance 128 is acquired as the positional relationship between the plurality of positions from the upstream side to the downstream side in the virtual bronchus (see FIG. 10). After the process of step ST38 is executed, the virtual bronchial moving image display process proceeds to step ST40.

In step ST40, the third display control unit 70D displays the virtual bronchial moving image 116 on the second screen 24 in the display aspect corresponding to the positional relationship acquired in step ST38 (see FIG. 11). For example, the third display control unit 70D advances the display of the virtual bronchial moving image 116 at the speed-along-pathway 130 calculated based on the first distance 128 and the movement required time 132 (see FIG. 11). After the process of step ST40 is executed, the virtual bronchial moving image display process proceeds to step ST42.

In step ST42, the positional relationship acquisition unit 70F determines whether or not a condition for ending the virtual bronchial moving image display process (hereinafter, referred to as a “virtual bronchial moving image display process end condition”) is satisfied. Examples of the virtual bronchial moving image display process end condition include a condition that an instruction to end the virtual bronchial moving image display process is received by the reception device. In step ST42, in a case where the virtual bronchial moving image display process end condition is not satisfied, a negative determination is made, and the virtual bronchial moving image display process proceeds to step ST36. In step ST42, in a case where the virtual bronchial moving image display process end condition is satisfied, an affirmative determination is made, and the virtual bronchial moving image display process proceeds to step ST44.

In step ST44, the third display control unit 70D ends the display of the virtual bronchial moving image 116 on the second screen 24. After the process of step ST44 is executed, the virtual bronchial moving image display process ends.

As described above, in the endoscope system 10, the virtual bronchial moving image 116 generated based on the pathway-inclusive bronchial volume data 100 is displayed on the second screen 24 of the display device 14. The doctor 16 operates the bronchoscope 18 with reference to the virtual bronchial moving image 116 displayed on the second screen 24, and moves the distal end part 36 of the bronchoscope 18 to the back side of the bronchus 66 along the pathway 68. Since the captured bronchial moving image 122 obtained by imaging the inside of the bronchus 66 via the endoscope 38 is displayed on the first screen 22 of the display device 14, the doctor 16 can compare the captured bronchial moving image 122 with the virtual bronchial moving image 116.

The doctor 16 gives the voice instruction to the control device 46 in a case where the doctor 16 determines that the adjustment of the display aspect of the virtual bronchial moving image 116 is necessary while comparing the captured bronchial moving image 122 with the virtual bronchial moving image 116. In the endoscope system 10, the positional relationship between the plurality of positions from the upstream side to the downstream side in the virtual bronchus (for example, the position corresponding to the current frame on the bronchial pathway 98A and the position corresponding to the voice instruction frame) is acquired according to the voice instruction given from the doctor 16. Then, the virtual bronchial moving image 116 is displayed in the display aspect corresponding to the positional relationship between the plurality of positions from the upstream side to the downstream side in the virtual bronchus. Since the plurality of positions from the upstream side to the downstream side in the virtual bronchus are acquired according to the voice instruction given by the doctor 16, the display aspect corresponding to the positional relationship between the plurality of positions from the upstream side to the downstream side in the virtual bronchus can be said to be a display aspect determined according to the voice instruction given by the doctor 16, so to speak. Therefore, with the present configuration, the virtual bronchial moving image 116 can be displayed in the display aspect convenient for the doctor 16. As a result, the operation of the bronchoscope 18 performed by the doctor 16 is supported by the display of the virtual bronchial moving image 116 as intended by the doctor 16. With the endoscope system 10, since the virtual bronchial moving image 116 is displayed in the display aspect convenient for the doctor 16, even in the bronchoscope 18 in which a system (for example, an electromagnetic navigation system or the like) that detects the position of the distal end part 36 is not implemented, the virtual bronchial moving image 116 corresponding to the position of the frame of the captured bronchial moving image 122 (that is, the portion of the distal end part 36 imaged by the endoscope 38) can be easily displayed.

In the endoscope system 10, the position of the upstream bifurcation in the virtual bronchus and the position of the downstream bifurcation are acquired according to the voice instruction given by the doctor 16. Then, the virtual bronchial moving image 116 is displayed in the display aspect corresponding to the positional relationship between the position of the upstream bifurcation in the virtual bronchus and the position of the downstream bifurcation. Since the position of the upstream bifurcation in the virtual bronchus and the position of the downstream bifurcation are acquired according to the voice instruction given by the doctor 16, the display aspect corresponding to the positional relationship between the position of the upstream bifurcation in the virtual bronchus and the position of the downstream bifurcation can be said to be the display aspect determined according to the voice instruction given by the doctor 16, so to speak. Therefore, with the present configuration, the moving image can be displayed in the display aspect convenient for the doctor 16 between the position of the upstream bifurcation in the virtual bronchus and the position of the downstream bifurcation.

In the endoscope system 10, the positional relationship between the position corresponding to the upstream bifurcation in the virtual bronchus and the position corresponding to the downstream bifurcation is defined by using the first distance 128. Then, the virtual bronchial moving image 116 is displayed in the display aspect corresponding to the first distance 128. Therefore, with the present configuration, the doctor 16 can understand the aspect in the bronchus 66 through the virtual bronchial moving image 116 displayed in the display aspect corresponding to the first distance.

In the endoscope system 10, the display of the virtual bronchial moving image 116 is advanced at the speed-along-pathway 130 determined according to the movement required time 132 and the first distance 128. Therefore, with the present configuration, the doctor 16 can observe the plurality of frames 118 corresponding between the plurality of positions (for example, between the position corresponding to the current frame on the bronchial pathway 98A and the position corresponding to the voice instruction frame) in the virtual bronchial moving image 116 displayed on the second screen 24 at a pace convenient for the doctor 16.

In the endoscope system 10, the plurality of bronchial pathways 98 are obtained by thinning the bronchial volume data 96. Then, the aspect in which the inside of the virtual bronchus is observed from the viewpoint 117 along the bronchial pathway 98A selected by the user from among the plurality of bronchial pathways 98 is displayed on the second screen 24 as the virtual bronchial moving image 116. Therefore, with the present configuration, the doctor 16 can continuously observe an aspect close to the aspect in which the inside of the bronchus 66 is observed from the endoscope 38 of the bronchoscope 18 along the pathway 68, through the virtual bronchial moving image 116 displayed on the second screen 24.

In the endoscope system 10, the display of the virtual bronchial moving image 116 is advanced at the constant speed along the bronchial pathway 98A between the plurality of positions from the upstream side to the downstream side in the virtual bronchus (for example, between the position corresponding to the current frame on the bronchial pathway 98A and the position corresponding to the voice instruction frame). Therefore, with the present configuration, it is possible to prevent the doctor 16 who observes the virtual bronchial moving image 116 from experiencing visual discomfort caused by a sudden change in the speed at which the display of the virtual bronchial moving image 116 is advanced.

### First Modification Example

In the above-described embodiment, as an example of the plurality of positions from the upstream side to the downstream side in the virtual bronchus, the position of the upstream bifurcation and the position of the downstream bifurcation have been described, but the technology of the present disclosure is not limited to this. For example, a position upstream of the upstream bifurcation in the virtual bronchus may be applied instead of the position of the upstream bifurcation, or a position upstream of the downstream bifurcation in the virtual bronchus may be applied instead of the position of the downstream bifurcation. Examples of the position upstream of the upstream bifurcation in the virtual bronchus include a nearest position at which a plurality of holes (for example, two holes) separated by the upstream bifurcation can be included in a visual field (virtual angle of view) from the viewpoint 117. Examples of the position upstream of the downstream bifurcation in the virtual bronchus include a nearest position at which a plurality of holes (for example, two holes) separated by the downstream bifurcation can be included in the visual field (virtual angle of view) from the viewpoint 117.

As described above, by using the position upstream of the upstream bifurcation in the virtual bronchus and the position upstream of the downstream bifurcation in the virtual bronchus as the plurality of positions from the upstream side to the downstream side in the virtual bronchus, the virtual bronchial moving image 116 is displayed in the display aspect corresponding to the positional relationship between the position upstream of the upstream bifurcation in the virtual bronchus and the position upstream of the downstream bifurcation in the virtual bronchus. Therefore, it is possible to suppress a phenomenon in which the plurality of holes separated by the bifurcations cannot be observed because the observation position in the virtual bronchus is too close to the upstream bifurcation and the downstream bifurcation in the virtual bronchial moving image 116 displayed on the second screen 24.

### Second Modification Example

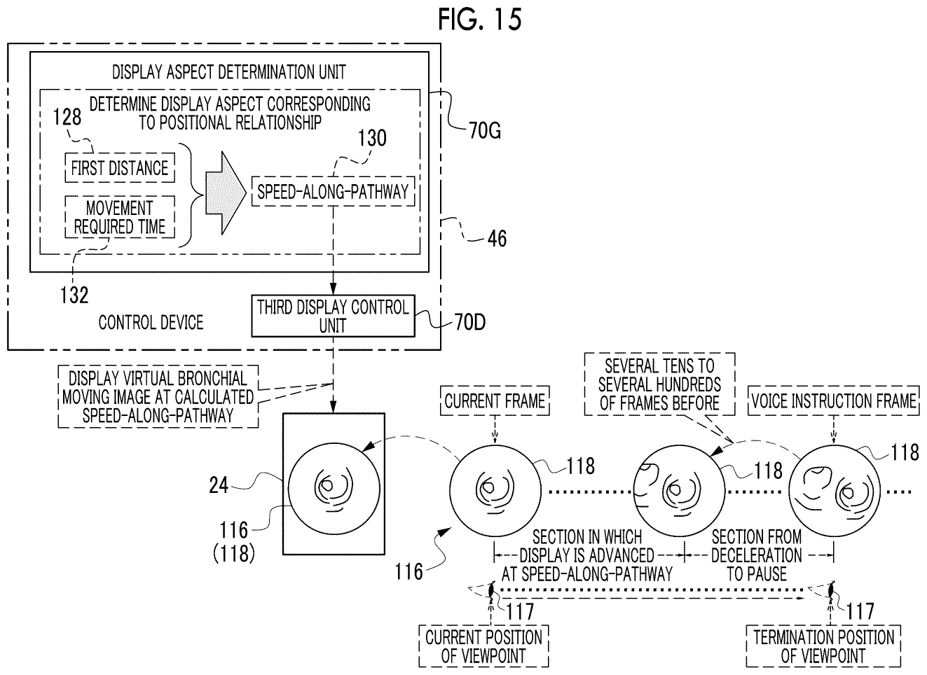

In the above-described embodiment, the example in which the third display control unit 70D advances the display from the current frame in the virtual bronchial moving image 116 to the voice instruction frame at the speed-along-pathway 130 has been described, but the technology of the present disclosure is not limited to this. For example, as illustrated in FIG. 15, in a section from the current frame in the virtual bronchial moving image 116 to several tens to several hundreds of frames before the voice instruction frame, the display may be advanced at the speed-along-pathway 130, and in a section from several tens to several hundreds of frames before the voice instruction frame to the voice instruction frame, the speed at which the display of the virtual bronchial moving image 116 is advanced is decreased gradually (for example, in a multi-stage or continuous manner), and the display of the virtual bronchial moving image 116 is stopped (that is, paused) at a point in time at which the voice instruction frame is reached.

That is, the speed at which the viewpoint 117 is advanced along the bronchial pathway 98A from the current frame to the middle is set as the speed-along-pathway 130, and the speed from the middle to the voice instruction frame is set to a speed slower than the speed-along-pathway 130. Then, at a timing at which the viewpoint 117 reaches the termination position, that is, the voice instruction frame, the speed at which the viewpoint 117 is advanced along the bronchial pathway 98A is set to “0”. As a result, the frame 118 in the vicinity of the voice instruction frame in the virtual bronchial moving image 116 displayed on the second screen 24 can be observed by the doctor 16 over time.

It should be noted that the display of the virtual bronchial moving image 116 may be stopped (that is, paused) at the position upstream of the bifurcation in the virtual bronchus. In this way, the doctor 16 can observe a vicinity of the bifurcation in the virtual bronchus over time.

### Third Modification Example

In the above-described embodiment, as the display aspect of the virtual bronchial moving image 116, the form example in which the display of the virtual bronchial moving image 116 is advanced along the bronchial pathway 98A has been described, but the technology of the present disclosure is not limited to this. For example, since the doctor 16 may rotate the distal end part 36 of the bronchoscope 18 around the pathway 68 in the bronchus 66, the virtual bronchial moving image 116 may be rotated around the bronchial pathway 98A in response to such a movement.

Examples of the scenario in which the distal end part 36 of the bronchoscope 18 is rotated around the pathway 68 in the bronchus 66 include a scenario in which the distal end part 36 reaches the vicinity of the bifurcation of the bronchus 66. In this scenario, in order to facilitate the operation of the bronchoscope 18 (for example, in order to facilitate the insertion of the distal end part 36 into the bifurcation hole), the doctor 16 may rotate the distal end part 36 around the pathway 68 such that an opening of the bronchus 66 is located at an upper part of the screen. In a case where the distal end part 36 reaches the vicinity of the bifurcation of the bronchus 66 and then the distal end part 36 is rotated around the pathway 68, the third display control unit 70D rotates the virtual bronchial moving image 116 around the bronchial pathway 98A at a timing at which the viewpoint 117 reaches the position of the voice instruction frame or in the vicinity of the voice instruction frame.

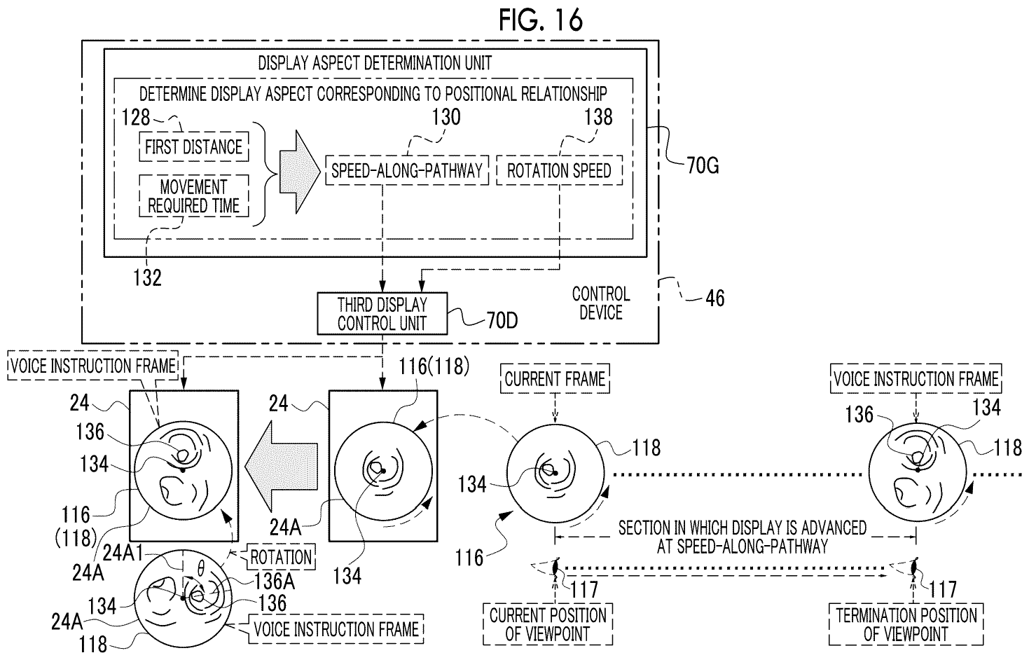

The distal end part 36 may be gradually rotated around the pathway 68 in a process of advancing the distal end part 36 along the pathway 68, instead of rotating the distal end part 36 around the pathway 68 after the distal end part 36 reaches the vicinity of the bifurcation of the bronchus 66. In this case, the third display control unit 70D gradually rotates the virtual bronchial moving image 116 around the bronchial pathway 98A as the viewpoint 117 is moved toward the termination position. In the third modification example, a form example in which the virtual bronchial moving image 116 is gradually rotated around the bronchial pathway 98A will be described with reference to FIG. 16.

As illustrated in FIG. 16 as an example, the display aspect of the virtual bronchial moving image 116 includes a rotational display aspect in which the virtual bronchial moving image 116 is rotationally displayed around a rotation axis 134. For example, in this case, the third display control unit 70D rotationally displays the virtual bronchial moving image 116 around the rotation axis 134 according to the positional relationship (for example, the positional relationship between the position of the current frame and the position of the voice instruction frame) obtained according to the voice instruction given from the doctor 16. The rotation axis 134 is an axis corresponding to the bronchial pathway 98A. That is, the rotation axis 134 is obtained by thinning the bronchial volume data 96.

In the second screen 24, a display region 24A in which the virtual bronchial moving image 116 is displayed is a circular region and is located in a central portion of the second screen 24. The virtual bronchial moving image 116 is rotated around the rotation axis 134 such that an opening portion image region 136 showing an opening portion of the bifurcation included in the virtual bronchus is located on an upper side in a front view of the display region 24A.

In the example illustrated in FIG. 16, the display aspect determination unit 70G calculates a rotation speed 138. The rotation speed 138 is a speed at which the virtual bronchial moving image 116 is rotationally displayed around the rotation axis 134. The rotation speed 138 is determined according to the positional relationship acquired by the positional relationship acquisition unit 70F (see FIG. 10). For example, the rotation speed 138 is determined according to the first distance 128 and the movement required time 132. Here, the first distance 128 is an example of a “second distance” according to the technology of the present disclosure.

The display aspect determination unit 70G calculates the rotation speed 138 based on the first distance 128 and the movement required time 132. For example, the rotation speed 138 is a constant speed and is calculated as a speed at which the opening portion image region 136 is located on the upper side in the front view of the display region 24A from a time at which the current frame is displayed on the second screen 24 to a time at which the voice instruction frame is displayed.

The third display control unit 70D rotationally displays the virtual bronchial moving image 116 around the rotation axis 134 at the rotation speed 138 as the viewpoint 117 is moved to the position of the voice instruction frame at the speed-along-pathway 130 along the bronchial pathway 98A, to rotate the opening portion image region 136 toward the upper side in the front view of the display region 24A. The opening portion image region 136 related to the voice instruction frame is rotated by a rotation amount corresponding to an angle θ about the rotation axis 134 during a period in which the viewpoint 117 is moved along the bronchial pathway 98A at the speed-along-pathway 130 to the position of the voice instruction frame. The angle θ is an angle between a position 136A of the opening portion image region 136 and a target position 24A1 on the upper side in the front view of the display region 24A. In the voice instruction frame, the position 136A is defined by a line segment connecting the rotation axis 134 and a circumference of the display region 24A through a centroid of the opening portion image region 136. The target position 24A1 is defined by a line segment connecting the rotation axis 134 and an upper semicircular point of the display region 24A.

The third display control unit 70D rotationally displays the virtual bronchial moving image 116 around the rotation axis 134, to locate the opening portion image region 136 at the target position 24A1 at the timing at which the viewpoint 117 reaches the termination position (that is, the position corresponding to the voice instruction frame). As a result, the opening portion image region 136 is located on the upper side in the front view of the display region 24A.

As described above, in the third modification example, the display aspect of the virtual bronchial moving image 116 includes the rotational display aspect in which the virtual bronchial moving image 116 is rotationally displayed around the rotation axis 134. The rotational display aspect is an aspect in which the virtual bronchial moving image 116 is rotationally displayed around the rotation axis 134 according to the positional relationship (for example, the positional relationship between the position of the current frame and the position of the voice instruction frame) obtained according to the voice instruction given by the doctor 16. Therefore, with the present configuration, a portion of interest (for example, the opening portion image region 136) in the virtual bronchus can be aligned with the position convenient for the doctor 16 in the virtual bronchial moving image 116 displayed on the second screen 24.

In the third modification example, the virtual bronchial moving image 116 is rotated around the rotation axis 134 obtained by thinning the bronchial volume data 96. That is, the axis corresponding to the bronchial pathway 98A is used as the rotation axis 134, and the virtual bronchial moving image 116 is rotated around the rotation axis 134. Therefore, with the present configuration, it is possible to stabilize the rotation pathway for the rotational display of the virtual bronchial moving image 116.

In the third modification example, the display aspect determination unit 70G calculates the rotation speed 138. The rotation speed 138 is the speed at which the virtual bronchial moving image 116 is rotationally displayed around the rotation axis 134. The rotation speed 138 is determined according to the positional relationship acquired by the positional relationship acquisition unit 70F (see FIG. 10). Since the plurality of positions from the upstream side to the downstream side in the virtual bronchus are acquired according to the voice instruction given by the doctor 16, the rotation speed 138 determined according to the positional relationship (that is, the positional relationship acquired by the positional relationship acquisition unit 70F) between the plurality of positions from the upstream side to the downstream side in the virtual bronchus can be said to be a rotation speed determined according to the voice instruction given by the doctor 16, so to speak. Therefore, with the present configuration, the portion of interest (for example, the opening portion image region 136) to the doctor 16 in the virtual bronchial moving image 116 displayed on the second screen 24 can be rotated at the pace convenient for the doctor 16.

In the third modification example, the rotational display of the virtual bronchial moving image 116 is advanced at the rotation speed 138 determined according to the movement required time 132 and the first distance 128. Therefore, with the present configuration, the pace at which the display is advanced along the bronchial pathway 98A between the plurality of positions (for example, between the current frame and the voice instruction frame) in the virtual bronchial moving image 116 displayed on the second screen 24 can be matched with the pace at which the portion of interest (for example, the opening portion image region 136) to the doctor 16 is rotated.

In addition, in the third modification example, the third display control unit 70D rotationally displays the virtual bronchial moving image 116 around the rotation axis 134 at the rotation speed 138 as the viewpoint 117 is moved along the bronchial pathway 98A at the speed-along-pathway 130 to the position of the voice instruction frame, so that the opening portion image region 136 is rotated toward the upper side in the front view of the display region 24A. Therefore, with the present configuration, the doctor 16 can sense an aspect in which the opening portion image region 136 is rotated toward the upper side in the front view of the display region 24A in a sensation close to a sensation in which the bronchoscope 18 is actually inserted into the body.

In addition, in the third modification example, during a period in which the viewpoint 117 is moved along the bronchial pathway 98A at the speed-along-pathway 130 to the position of the voice instruction frame, the opening portion image region 136 related to the voice instruction frame is rotated by the rotation amount corresponding to the angle θ about the rotation axis 134. The angle θ is the angle between the position 136A of the opening portion image region 136 and the target position 24A1 on the upper side in the front view of the display region 24A. Therefore, with the present configuration, the doctor 16 can sense an aspect in which the opening portion image region 136 is rotated toward the target position 24A1 on the upper side in the front view of the display region 24A in the sensation close to the sensation in which the bronchoscope 18 is actually inserted into the body.

In addition, in the third modification example, the third display control unit 70D rotationally displays the virtual bronchial moving image 116 around the rotation axis 134, to locate the opening portion image region 136 at the target position 24A1 at the timing at which the viewpoint 117 reaches the termination position (that is, the position corresponding to the voice instruction frame). Therefore, with the present configuration, the opening portion image region 136 can be observed by the doctor 16 on the upper side in the front view of the display region 24A at the timing at which the viewpoint 117 reaches the termination position.

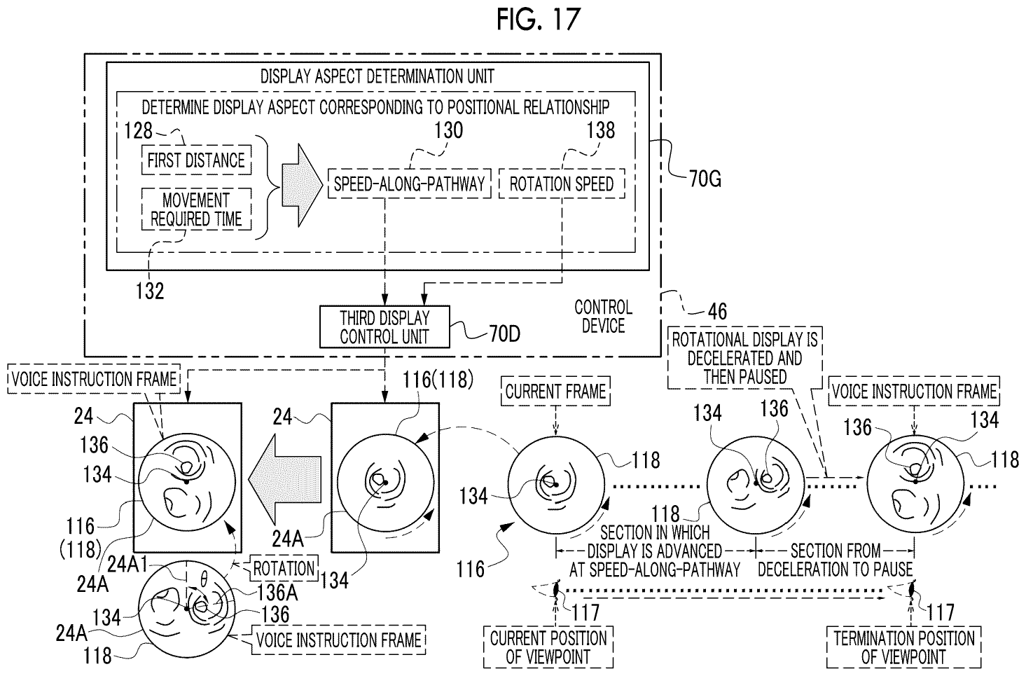

In the example illustrated in FIG. 16, the case where the rotational display speed from the current frame to the voice instruction frame (that is, the rotation speed 138) is a constant speed has been described, but the technology of the present disclosure is not limited to this. For example, as illustrated in FIG. 17, in a section from the deceleration of the viewpoint 117 to the pause of the viewpoint 117, the third display control unit 70D may also decelerate the rotational display of the virtual bronchial moving image 116 according to the speed at which the viewpoint 117 is moved along the bronchial pathway 98A, and stop the rotational display at the termination position of the viewpoint 117.

### Fourth Modification Example

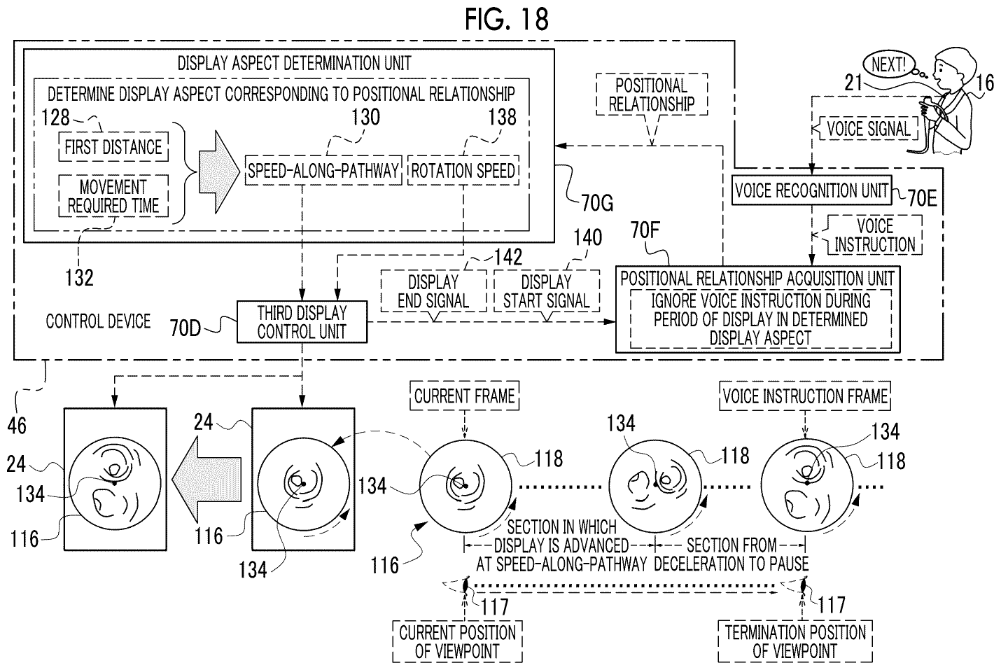

As illustrated in FIG. 18 as an example, the positional relationship acquisition unit 70F ignores the voice instruction received from the voice recognition unit 70E (that is, the voice instruction given by the doctor 16) during a period in which the virtual bronchial moving image 116 is displayed on the second screen 24 in the display aspect determined by the display aspect determination unit 70G. The period in which the virtual bronchial moving image 116 is displayed on the second screen 24 in the display aspect determined by the display aspect determination unit 70G is specified by a display start signal 140 and a display end signal 142.

The display start signal 140 is a signal indicating that the display of the virtual bronchial moving image 116 in the display aspect determined by the display aspect determination unit 70G is started. The display end signal 142 is a signal indicating that the display of the virtual bronchial moving image 116 in the display aspect determined by the display aspect determination unit 70G ends. The third display control unit 70D outputs the display start signal 140 to the positional relationship acquisition unit 70F in a case where the display of the virtual bronchial moving image 116 in the display aspect determined by the display aspect determination unit 70G is started. The third display control unit 70D outputs the display end signal 142 to the positional relationship acquisition unit 70F in a case where the display of the virtual bronchial moving image 116 in the display aspect determined by the display aspect determination unit 70G ends.

The positional relationship acquisition unit 70F ignores the voice signal from the input of the display start signal 140 to the input of the display end signal 142. As a result, it is possible to prevent the display aspect of the virtual bronchial moving image 116 from being changed at a timing unintended by the doctor 16.

It should be noted that, here, the form example in which the positional relationship acquisition unit 70F ignores the voice signal has been described, but this is merely an example, and the positional relationship acquisition unit 70F may not receive the voice instruction. The voice recognition unit 70E may not receive the voice signal, or the voice recognition unit 70E may ignore the voice signal. The power of the microphone 21 may be turned off, or the microphone 21 may be set to a sleep mode.

### Other Modification Examples

In the above-described embodiment, the form example in which the frame 118 (that is, the voice instruction frame) including the bifurcation to be displayed next in the virtual bronchial moving image 116 as the image is selected according to the voice instruction (for example, the voice instruction “next”) has been described, but the technology of the present disclosure is not limited to this. For example, the frame 118 including a plurality of bifurcations as the images in the virtual bronchial moving image 116 may be selected by the voice instruction (for example, a voice instruction “N bifurcations ahead” in a case where Nis a natural number of 2 or more). In addition, for example, the frame 118 corresponding to the bifurcation may be selected by the voice instruction using names given in advance to the plurality of bifurcations. The frame 118 corresponding to at least one portion on the bronchial pathway 98A other than the bifurcation (for example, between the bifurcation of #2 and the bifurcation of #3) may be selected by the voice instruction (for example, a voice instruction “center position between #2 and #3”, a voice instruction “xx millimeters ahead”, or a voice instruction “vicinity of the front of next bifurcation”).

In the above-described embodiment, the form example in which the speed-along-pathway 130 and the rotation speed 138 are calculated by the display aspect determination unit 70G using one first distance 128 has been described, but the technology of the present disclosure is not limited to this. For example, a plurality of the speeds-along-pathway 130 and a plurality of rotation speeds 138 may be calculated by the display aspect determination unit 70G using a plurality of first distances 128. For example, in this case, the first distance 128 may be calculated between a plurality of portions selected according to the voice instruction in the bronchial pathway 98A.

Examples of “between the plurality of portions” include “between the plurality of bifurcations in the virtual bronchus”. For example, “between the plurality of bifurcations” means a plurality of sections selected by the voice instruction from a section from the start point of the bronchial pathway 98A illustrated in FIG. 10 to “#1”, a section from “#1” to “#2”, a section from “#2” to “#3”, a section from “#3” to “#4”, and a section from “#5” to the end point of the bronchial pathway 98A. As described above, in a case where the viewpoint 117 is moved across the plurality of sections, the opening portion image region 136 may be located on the upper side in the front view of the display region 24A as in the examples illustrated in FIGS. 16 and 17.

In the above-described embodiment, the bronchoscope 18 has been described as an example, but the technology of the present disclosure is not limited to this, and the technology of the present disclosure is established even in an endoscope that observes a cavity region (for example, a region from the esophagus to the duodenum, a region from the anus to the small intestine, or the like) in the body, such as an upper digestive organ endoscope or a lower digestive organ endoscope. In this case, the cavity region in the body corresponds to the trachea 64 and the bronchus 66 to which the pathway 68 described in the above-described embodiment is assigned.

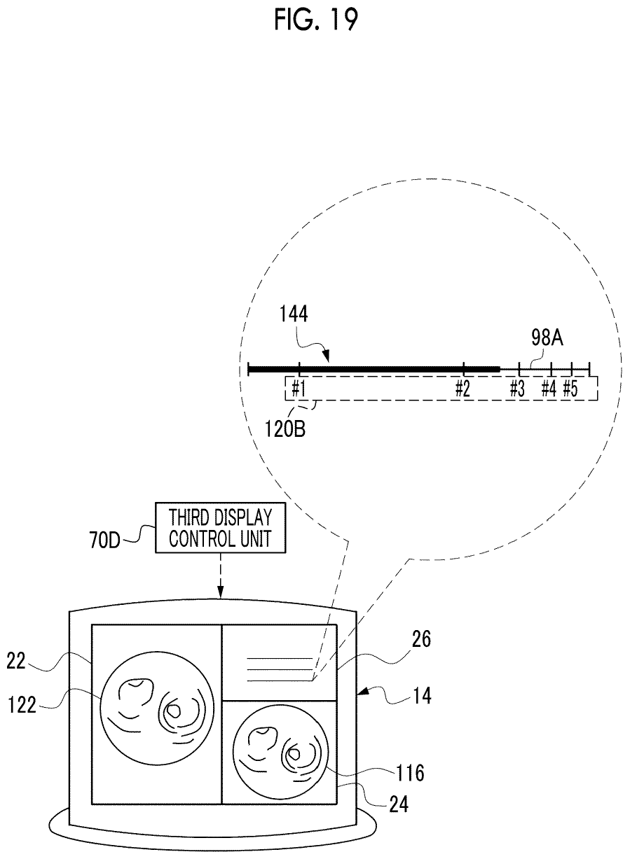

In the above-described embodiment, it is possible to visually understand at which position on the bronchial pathway 98A the viewpoint 117 is located (that is, the current position of the viewpoint 117) from the virtual bronchial moving image 116 displayed on the second screen 24. However, for example, as illustrated in FIG. 19, the current position of the viewpoint 117 may be understood by using a seek bar 144. In the example illustrated in FIG. 19, the third display control unit 70D displays the seek bar 144 on the third screen 26. In the seek bar 144, the bronchial pathway 98A is illustrated in a linear shape, and the bifurcation identifier 120B is assigned to the bronchial pathway 98A. In addition, in the seek bar 144, a portion in the bronchial pathway 98A through which the viewpoint 117 passes is displayed in an aspect distinguishable from the other portions. It should be noted that a display location of the seek bar 144 need not be on the third screen 26, and may be on the first screen 22, the second screen 24, or a screen of another display device. An indicator for visually identifying the current position of the viewpoint 117 may be used instead of the seek bar 144.

In the above-described embodiment, the form example in which the first screen 22, the second screen 24, and the third screen 26 are displayed on the display device 14 has been described, but the first screen 22, the second screen 24, and the third screen 26 may be dispersively displayed by different display devices. A size of the first screen 22, a size of the second screen 24, and a size of the third screen 26 may be selectively changed.

In the above-described embodiment, the form example in which the virtual bronchial moving image acquisition process, the endoscopic image display process, and the virtual bronchial moving image display process (hereinafter, these processes will be referred to as “various processes”) are performed by the processor 70 of the endoscope apparatus 12 has been described, but the technology of the present disclosure is not limited to this. For example, a device that performs the various processes may be provided outside the endoscope apparatus 12. Examples of the device provided outside the endoscope apparatus 12 include a server. For example, the server is implemented by cloud computing. Here, cloud computing has been described as an example, but this is merely an example. For example, the server may be implemented by a mainframe or may be implemented by network computing such as fog computing, edge computing, or grid computing. Here, the server has been described as an example of the device provided outside the endoscope apparatus 12, but this is merely an example. For example, at least one personal computer may be used instead of the server. In addition, various processes may be dispersively performed by a plurality of devices including the endoscope apparatus 12 and the device provided outside the endoscope apparatus 12.

In the above-described embodiment, the form example in which the virtual bronchial moving image acquisition program 84, the endoscopic image display program 86, and the virtual bronchial moving image display program 88 (hereinafter, these programs will be referred to as “various programs”) are stored in the NVM 74 has been described, but the technology of the present disclosure is not limited to this. For example, various programs may be stored in a portable storage medium, such as an SSD or a USB memory. The storage medium is a non-transitory computer-readable storage medium. The various programs stored in the storage medium are installed in the computer 69 of the control device 46. The processor 70 executes various processes according to the various programs.

In the above-described embodiment, the computer 69 has been described, but the technology of the present disclosure is not limited to this, and a device including an ASIC, an FPGA, and/or a PLD may be applied instead of the computer 69. Also, a combination of a hardware configuration and a software configuration may be used instead of the computer 69.

The following various processors can be used as hardware resources for executing each of the various processes described in the above-described embodiment. Examples of the processor include a processor as a general-purpose processor that executes software, that is, a program, to function as the hardware resource executing the various processes. Examples of the processor also include a dedicated electronic circuit as a processor having a dedicated circuit configuration designed to execute a specific process, such as an FPGA, a PLD, or an ASIC. Any processor includes a memory built therein or connected thereto, and any processor uses the memory to execute the various processes.

The hardware resource for executing the various processes may be configured by one of the various processors or by a combination of two or more processors of the same type or different types (for example, a combination of a plurality of FPGAs or a combination of a processor and an FPGA). Further, the hardware resource for executing the various processes may be one processor.

A first example of the configuration in which the hardware resource is configured by one processor is an aspect in which one processor is configured by a combination of one or more processors and software and functions as the hardware resource for executing the various processes. A second example of the configuration is an aspect in which a processor that implements the functions of the entire system including a plurality of hardware resources for executing the various processes using one IC chip is used. A representative example of this aspect is an SoC. As described above, the various processes are implemented by using one or more of the various processors as the hardware resource.

Further, specifically, an electronic circuit obtained by combining circuit elements, such as semiconductor elements, can be used as the hardware structure of the various processors. The various processes are merely examples. Therefore, it goes without saying that unnecessary steps may be deleted, new steps may be added, or the process order may be changed, without departing from the gist.

The contents described and illustrated above are detailed descriptions of portions related to the technology of the present disclosure and are merely examples of the technology of the present disclosure. For example, the descriptions of the configurations, functions, operations, and effects are the descriptions of examples of the configurations, functions, operations, and effects of the portions related to the technology of the present disclosure. Therefore, it goes without saying that unnecessary portions may be deleted or new elements may be added or replaced in the contents described and illustrated above, without departing from the gist of the technology of the present disclosure. In addition, the description of, for example, common technical knowledge that does not need to be particularly described to enable the implementation of the technology of the present disclosure is omitted in the contents described and illustrated above in order to avoid confusion and to facilitate the understanding of the portions related to the technology of the present disclosure.

In the present specification, “A and/or B” is synonymous with “at least one of A or B”. That is, “A and/or B” means that it may be only A, only B, or a combination of A and B. In addition, in the present specification, in a case where three or more matters are associated and expressed by “and/or”, the same concept as “A and/or B” is applied.

All of the documents, the patent applications, and the technical standards described in the specification are incorporated by reference herein to the same extent as each individual document, each patent application, and each technical standard is specifically and individually stated to be incorporated by reference.

What is claimed is:

1. An image processing apparatus comprising:
a processor,
wherein the processor
displays, on a display device, a moving image that is generated based on volume data including a bronchial image showing a bronchus and that shows an aspect in which an inside of the bronchus is observed,
receives an instruction performed by a voice,
acquires a positional relationship between a plurality of positions from an upstream side to a downstream side in the bronchus according to the instruction, and
displays the moving image in a display aspect corresponding to the positional relationship.

2. The image processing apparatus according to claim 1,
wherein the plurality of positions include a position corresponding to an upstream bifurcation in the bronchus and a position corresponding to a downstream bifurcation in the bronchus.

3. The image processing apparatus according to claim 2,
wherein the position corresponding to the upstream bifurcation is a position upstream of the upstream bifurcation in the bronchus, and
the position corresponding to the downstream bifurcation is a position upstream of the downstream bifurcation in the bronchus.

4. The image processing apparatus according to claim 3,
wherein the display aspect includes an aspect in which the display of the moving image is stopped at the position corresponding to the upstream bifurcation and at the position corresponding to the downstream bifurcation, an aspect in which a speed at which the display of the moving image is advanced is decreased, or an aspect in which the display of the moving image is stopped after the speed at which the display of the moving image is advanced is decreased.

5. The image processing apparatus according to claim 1,
wherein the positional relationship is defined by a first distance that is a distance between the plurality of positions.

6. The image processing apparatus according to claim 5,
wherein the display aspect includes an aspect in which the display of the moving image is advanced at a speed determined according to a time required for movement between the plurality of positions and the first distance.

7. The image processing apparatus according to claim 1,
wherein the display aspect includes a rotational display aspect in which the moving image is rotationally displayed around a rotation axis determined for the moving image according to the positional relationship.

8. The image processing apparatus according to claim 7,
wherein the rotation axis is obtained by thinning the bronchial image.

9. The image processing apparatus according to claim 7,
wherein a speed at which the moving image is rotationally displayed is determined according to the positional relationship.

10. The image processing apparatus according to claim 9,
wherein the positional relationship is defined by a second distance that is a distance between the plurality of positions, and
the speed at which the moving image is rotationally displayed is determined according to the second distance and a time required for movement between the plurality of positions.

11. The image processing apparatus according to claim 7,
wherein the moving image shows an aspect in which the inside of the bronchus is observed from a viewpoint in the bronchus, and
the rotational display aspect is an aspect in which the moving image is rotationally displayed around the rotation axis in response to movement of the viewpoint between the plurality of positions, to rotate an opening portion image region showing an opening portion of a bifurcation included in the bronchus toward an upper side in a front view of the display region in which the moving image is displayed.

12. The image processing apparatus according to claim 11,
wherein the rotational display aspect is an aspect in which the opening portion image region is rotated by a rotation amount corresponding to an angle about the rotation axis between a position of the opening portion image region and a target position on the upper side in the front view.

13. The image processing apparatus according to claim 11,
wherein the rotational display aspect is an aspect in which the moving image is rotationally displayed around the rotation axis, to locate the opening portion image region on the upper side in the front view at a timing at which the viewpoint reaches a termination position among the plurality of positions.

14. The image processing apparatus according to claim 1,
wherein the processor does not receive the instruction or ignores the received instruction during a period in which the moving image is displayed in the display aspect.

15. The image processing apparatus according to claim 1,
wherein the moving image shows an aspect in which the inside of the bronchus is observed along a pathway obtained by thinning the bronchial image.

16. The image processing apparatus according to claim 15,
wherein the display aspect includes an aspect in which the display of the moving image is advanced at a constant speed along the pathway between the plurality of positions.

17. An endoscope apparatus comprising:
the image processing apparatus according to claim 1; and
an endoscope that acquires an image showing an aspect of the inside of the bronchus by imaging the inside of the bronchus and that outputs the acquired image.

18. An image processing method comprising:
displaying, on a display device, a moving image that is generated based on volume data including a bronchial image showing a bronchus and that shows an aspect in which an inside of the bronchus is observed;
receiving an instruction performed by a voice;
acquiring a positional relationship between a plurality of positions from an upstream side to a downstream side in the bronchus according to the instruction; and
displaying the moving image in a display aspect corresponding to the positional relationship.

19. A non-transitory computer-readable storage medium storing a program executable by a computer to perform a process comprising:
displaying, on a display device, a moving image that is generated based on volume data including a bronchial image showing a bronchus and that shows an aspect in which an inside of the bronchus is observed;
receiving an instruction performed by a voice;
acquiring a positional relationship between a plurality of positions from an upstream side to a downstream side in the bronchus according to the instruction; and
displaying the moving image in a display aspect corresponding to the positional relationship.
